(19) United States
(12) Patent Application Publication
(10) Pub. No.: US 2025/0295600 A1
(43) Pub. Date: Sep. 25, 2025

(54) EXTRACELLULAR VESICLES DERIVED FROM MILK AND PROCESS FOR ISOLATING THE SAME

(71) Applicant: EVOBIOTIX SA, Melide (CH)

(72) Inventors: Nicole C. Meisner-Kober, Salzburg (AT); Martin Hintersteiner-Wenzel, Oberndorf (AT); Raffaella Manzotti, Alta Valle Intelvi (CO) (IT); Martinus J.M. De Groot, Melide (CH)

(73) Assignee: EVOBIOTIX SA, Melide (CH)

(21) Appl. No.: 18/701,691

(22) PCT Filed: Oct. 18, 2022
(86) PCT No.: PCT/IB2022/059983

§ 371 Date: Apr. 16, 2024

(30) Foreign Application Priority Data
Oct. 22, 2021 (CH) CH070433/2021
Oct. 22, 2021 (IT) 102021000027167

(51) Int. Cl. A61K 9/50; A61K 9/00; A61K 31/7048; A61K 47/12

(52) U.S. Cl. CPC A61K 9/5068; A61K 9/0073; A61K 31/7048; A61K 47/12

(57) ABSTRACT

The present invention relates to the field of biotechnology, and particularly to milk derived extracellular vesicles, and provides a process for isolating such extracellular vesicles from milk and milk related fluids. The present invention is also related to compositions containing said extracellular vesicles derived from milk, particularly suitable for use in pharmaceutical, veterinary, cosmetic and/or nutraceutical applications.

This application is a U.S. national stage of PCT/IB2022/059983 filed 18 Oct. 2022, which claims priority to and the benefit of Swiss Patent Application No. CH070433/2021 filed 22 Oct. 2021, and which claims priority to and the benefit of Italian Patent Application No. 102021000027167 filed 22 Oct. 2021, the contents of which are incorporated herein by reference in their entireties.

### FIELD OF THE INVENTION

The present invention relates to the field of biotechnology, and particularly to milk derived extracellular vesicles (EVs). The invention provides a process for isolating such extracellular vesicles from milk and milk related fluids, which is especially suitable for industrial production due to its scalability and its ability to retain the structural integrity of the extracellular vesicles as well as its ability to provide solutions of homogenous purity, obtained purity being superior to previously known preparations in the field. The invention is further related to compositions of extracellular vesicles, derived from milk with said process, which show surprisingly high recovery/extraction yields and provides EVs which are particularly low in contaminating proteins and other contaminants, making them suitable for chemical modification e.g. by tagging with targeting molecules or loading with active pharmaceutical ingredients for use in pharmaceutical, veterinary, cosmetic and nutraceutical applications. The invention further relates to compositions of milk derived extracellular vesicles and methods of preparing the same, wherein the milk vesicles have been loaded, for example, with Amphotericin B or derivatives thereof, thereby providing potent antifungal, antiparasitic and anti-infective preparations for oral use.

The invention further provides a method for systemic delivery of milk EVs and their cargoes which results in surprisingly efficient systemic uptake and allows the delivery to multiple organs via the respiratory tract.

### TECHNICAL BACKGROUND

Extracellular Vesicles (EVs) are a known category of cell membrane derived components, comprising, among others, exosomes, ectosomes, apoptotic bodies and microvesicles. EVs, and in particular exosomes, have attracted enormous attention as part of an as yet incompletely understood long-distance cell-cell communication system opening up entirely new avenues for clinical biomarkers and diagnostics, cell-free cell therapy, vaccines and as potential drug carriers.

Exosomes are defined as double membrane enclosed particles, with a size of about 40-200 nm, derived from multivesicular bodies (MVBs), which are continuously produced and released by virtually all eukaryotic cell types into the extracellular space. Furthermore, exosomes have been shown to be present in a large range of body fluids, such as blood, saliva, urine as well as in the milk of mammals.

Compared to cell-culture derived exosomes, which are typically collected from the supernatant of cultured human or other higher organism cells, exosomes derived from natural sources used in the food industry (milk, fruit juices, etc.) hold the distinct advantage of being available at relatively high amounts with no need to establish specific biotechnological production methods, cellular bioreactors, and processes for their production. However, the concentrations at which exosomes are present in different starting materials is far below the useful levels for being directly suitable as drug carriers or therapeutic agents (as well as starting material of drug carriers or starting material for therapeutic agents), and the presence of additional components (such as casein and other proteins, lipoproteins, sugars or fatty acids) limits the downstream applications.

Consequently, there is a need for scalable methods for isolation and purification of exosomes from these attractive natural sources.

Over the past years, different processes for the isolation of cell-culture derived exosomes in the laboratory have been described in the literature. Such methods include many different procedures, such as, for example, differential and high-speed ultracentrifugation, centrifugation on density gradients, size exclusion chromatography, affinity chromatography, affinity-based adsorption using heparin, membrane-based separations, microfluidics or polyethylene glycol-based precipitation, as well as various commercialized isolation kits, such as e.g. ExoQuick™ (Antes, 2013).

It is important to notice that the above indicated isolation processes have mainly been reported by researchers conducting laboratory scale isolations of EVs for analytical, mechanistic or cell-based investigations, with relatively low volumes to be processed. Furthermore, these methods have mainly been applied to the isolation of cell-culture derived exosomes from supernatant, which contains a relatively low concentration of contaminating proteins and higher molecular weight impurities.

On the contrary, the isolation of exosomes from natural sources poses additional challenges; typical natural sources, such as milk, contain a high degree of dry matter and/or large amounts of fatty acids, sugars, other emulsifiers and/or contaminating proteins. Moreover, different starting materials represent different matrices with a wide range of potentially toxic, interacting, cross-reacting or interfering contaminants (e.g. other proteins, peptides, lipids, carbohydrates, oligonucleotides, such as micro-RNAs, or other natural products and derivatives). Consequently, each matrix poses specific and unique challenges for isolating EVs, or EV enriched fractions, of defined characteristics and constant, reproducible quality. This aspect is particularly important, because only such highly pure and reproducible EV preparations are suitable for further decoration, loading with drug molecules and/or modification for different targeting strategies, commonly employed in future generation drug delivery systems.

In the case of milk derived EVs, ultracentrifugation to collect the EVs in a pellet or defined fractions on density gradients is still the method of choice used for their isolation. However, aside from requiring specialized and expensive equipment, ultracentrifugation is technically difficult to scale-up, laborious and time-consuming. Furthermore, ultracentrifugation is separating molecular entities based on their relative densities, can lead to co-precipitation of aggregating proteins, lipoproteins and diverse oligonucleotides, such as small and microRNAs. Such contaminants can lead to significant overestimation of the actual yield of the preparation, especially when the evaluation is based on methods which determine the total protein content of a preparation. Additionally, ultracentrifugation via pelletisation inherently promotes the formation of exosome clusters and EV aggregates, potentially leading to vesicle fusions, distortions in size distributions and alterations in cell uptake and other biological activities. Finally, the ultracentrifugation process produces variable yields and qualities, making the process difficult to standardize.

Alternative methods known in the art are based on commercial kits using affinity-based isolation strategies, which are not only highly limited in scale and yields, but also require harsh conditions for elution of bound vesicles from the affinity resins with unknown consequences to EV integrity and function.

In a recent publication, Marsh et al. proposes, in order to purify EVs from bovine milk, two slightly different protocols both essentially based on the combined use of the complexing agent EDTA to solubilize casein micelles and final fractionation of the EVs via size exclusion chromatography after pre-purification either via ultracentrifugation or tangential flow filtration through a membrane with a molecular cut-off of 500 kDa. Therefore, both of these protocols require a chemical treatment of the milk using EDTA for the solubilization of casein aggregates and micelles. Furthermore, the EVs are recovered, at the end of the process, after a final passage through a Sepharose column (SEC). Those two steps (i.e. the use of EDTA and the SEC), together with the other process steps (ultracentrifuge or filtration), are described as mandatory, in order to obtain the desired quality of the final EV product. On the other hand, these steps by themselves impose additional limitation onto the process. Firstly, the use of EDTA solubilizes casein aggregates, increasing the amount of freely diffusing low molecular weight impurities that need to be eliminated with Tangential Flow Filtration (TFF). This leads to membrane fouling over time thereby limiting, as the authors comment themselves, the purity obtainable via TFF. Secondly, the need of using SEC fractionation to further purify the material after TFF, results in several different fractions of EV preparations of varying purity and compositions with different impurity levels, additionally limiting the scalability of the process.

Consequently, in the field of milk derived exosomes there is still the need for the development of an isolation process which avoids the drawbacks of ultracentrifugation and which is suitable to handle large volumes of starting fluid (i.e. milk), that can operate without specialized laboratory equipment and which allows to obtain exosome preparations of a homogenous, well defined and reproducible quality.

With respect to the use of EV preparations for drug delivery, special attention needs to be drawn not only to the EVs and their preparation, but also to the types of cargoes with which the EV delivery system is employed. Classical small molecules often exhibit an oral bioavailability on the order of 20-100%, whereas biomacromolecules and their derivatives, such as oligonucleotides and proteins, are considered not to be orally bioavailable. In general, such molecules, despite numerous attempts in the literature, are considered to be of use only for intravenous or subcutaneous injection or local applications. However, there are also numerous natural products with molecular characteristics generally described in the field of medicinal chemistry as not satisfying the “rules-of-five” criteria (Lipinsky et al. 2001, Adv. Drug Deliv. Rev. 46) and similar rule-based assessments and/or which do not show any therapeutically useful oral bioavailability. Now, several limited attempts have been conducted to load milk EVs with cargoes.

WO2018102397A1 describes milk EV preparations loaded with biomacromolecules. However, the exemplified loading procedures are limited to biomacromolecules mostly modified by a hydrophobic anchor, in order to allow a physical insertion or interaction of the macromolecular cargo with exosomes and/or by some sort of physico-mechanical disruption of the EV membranes via e.g. sonication or freeze-thaw cycles.

U.S. Pat. No. 10,420,723B2 discloses some preparations of milk EVs with a selection of natural products, the loading being affected either by suspending the drug in PEG-400 or using ethanol as co-solvent and, after co-incubation, separating EVs from the excess of free drug by using centrifugation and ultracentrifugation methods, such as those employed for isolation of EVs. Such a procedure suffers from the same limitations (scalability, yield, purity etc.) as the above commented procedures for the isolation of milk EVs, and has the additional drawback of unspecific drug binding to the non-vesicular proteins inherently present in EV samples, in particular when using ultracentrifugation-based processes for their isolation in the first place.

EP3620519A1, discloses the use of extrusion to load milk-EVs with hydrophobic anchor modified RNAs.

Such overtly generalized procedures do not automatically result in biologically active drug carriers but require laborious, case-by-case experimentation and are subject to identifying a workable combination by testing an unlimited list of additives, variables, and optimization parameters without an a priori guarantee of success. One example demonstrating that this principle still holds true for EVs and EV derived drug carrier systems is provided by Grossen et al. (Grossen et al., European Journal of Pharmaceutics and Biopharmaceutics, Volume 158, 2021), where no functional effect was observed after oral administration of RNA-loaded milk EVs as described in EP 3 620 519 A1.

Therefore, new and improved preparation methods for loading and application of milk EVs with specific cargo molecules are needed.

### Aims of the Invention

A first aim of the present invention is to provide a method for isolating EVs from milk of different mammalian species as is commonly produced in the dairy industry. Said isolated EVs having high purity and reproducible characteristics.

A further aim of the invention, is to provide a method for isolating EVs from colostrum of different species including human colostrum as well as from already processed milk of different species such as milk powders (produced by lyophilization or spray-drying) or pasteurized, defatted or otherwise processed milk and powders derived from such processed milk, which still contain at least partly intact EVs.

Another aim of the present invention is to provide EV preparations particularly suitable for further decoration and/or drug loading via methods described in the art.

Another aim of the present invention is to provide also new preparations of drug loaded milk EVs based on the antifungal and antiparasitic drug Amphotericin B (AmB).

Yet another aim of the present invention is to provide efficient methods for systemic delivery of EV preparations resulting in drug exposure in several different organs in mammals.

Those aims, among others, will be addressed in further details in the following paragraphs together with experimental data and examples, in order to fully clarify the subject matter of the present invention.

### SUMMARY OF THE INVENTION

The present invention addresses the foregoing and other needs, with respect to the prior art, by providing a new process suitable for the industrial isolation of exosomes from different milk starting materials. The present invention is also related to compositions, obtained by such isolation process, which are of high purity with respect to the possible presence of contaminating proteins, protein aggregates, aggregates of organic impurities and lipids, and to their use as nano-sized carriers for drug delivery. Furthermore, impurities can also derive from the microbiological characteristics of the starting material. In fact, milk is well known to represent a rich medium for microorganisms, and such contaminations are unavoidable in the process of milk production. Any manufacturing process for the isolation of EVs from a milk source needs to confront this difficulty.

The isolation method of the present invention is based on the surprising finding that a process consisting of a series of distinct steps, comprising an enzyme-based casein-coagulation step and a thermal treatment step, followed by ultrafiltration using membranes of specific pore sizes and ion-strength controlled dialysis, allows for a highly efficient isolation, purification and concentration of milk derived exosomes (EVs).

The surprising effect of the finding of the invention is related to the fact that protein and fat rich starting materials usually suffer from a lack of amenability to be highly concentrated, due to increasing aggregation, turbidity or precipitation. At the same time, dialysis with pure water can lead to depletion of stabilizing ions which are required to prevent protein aggregation. It is also important to take into consideration the fact that microbiologically rich natural matrices (i.e. milk) present a further obstacle during membrane based processing. In fact, the growth of the existing live microorganisms, and the presence of their metabolic and catabolic by-products, lead to membrane fouling and reduce the processing efficiency.

Furthermore, the process of the invention comprises a pre-determined series of purification steps, which result in the isolation of extracts, rich in EVs with average size ranges of 30-200 nm and which carry the surface-bound protein markers characteristic of exosomes. Said series of purification steps typically comprise a first phase, implying the coagulation of the majority of the milk protein components, followed by a heating step. Said heating step, also named pasteurization step in the present document, can assist the coagulation of the casein and, also, it is useful in reducing the bioburden (i.e. the microbiological contamination). It was surprisingly observed that, as will be demonstrated in the following experimental part, the viability of the EVs was not impacted by said thermal treatment. The coagulation step and the heating step are then followed by removal of the coagulated caseins via filtration, or by means of other separation methods. The second phase of the process implies the concentration and ion-strength controlled dialysis of the EVs fraction via ultrafiltration using membranes of specifically selected pore sizes.

In an embodiment of the present invention, following the isolation of EVs by means of the phases and steps above described, a further process can be carried out on the milk EVs isolate.

In particular, they can be frozen, with or without different cryoprotectants, lyophilized and/or spray-dried.

Therefore, the invention enables the isolation of large quantities of EVs, maintaining their intrinsic biological function to a degree comparable or superior to EVs isolated by means of the currently employed methods according to the prior art, which are typically based on differential ultracentrifugation or ultracentrifugation on density gradients, or a combination of other methods which are characterized, as already said, by several drawbacks, i.e. they are time-consuming, difficult to standardise, cumbersome and scale-limiting.

Although, technically, the first step of the process, i.e. the casein-coagulation, can be performed by an acid induced coagulation, this solution is less preferred with respect to an enzyme-based coagulation, due to functional consequences observed with the finally obtained preparations when acid is used. In fact, the EVs isolated employing the latter, exhibit on average larger particle sizes and a lower abundance of EV marker proteins such as TSG101 or CD9 in Western blots. Therefore, the acid induced casein coagulation seems to provide EV isolations which do not fulfil all the desired requirements.

Therefore, according to the present invention, the procedures for obtaining milk EV preparations largely devoid of non-vesicular protein contaminants, were applied to obtain, for example, Amphotericin B loaded milk EVs from two different species, bovine and goat. When comparing different loading conditions for Amphotericin B, it was surprisingly found that co-incubation for a specified amount of time in a specified ratio of Amphotericin B and milk-EVs provided functional Amphotericin B loaded EV preparations, containing the highest drug load of Amphotericin B with a stable association between drug and carrier. TFF-based dialysis with 750 kDa membranes allowed the ultracentrifugation free re-isolation of pure Amphotericin B loaded and biologically functional milk EVs. Such Amphotericin loaded milk EVs are suitable to treat certain medical conditions, in which the milk EVs as carrier provide the required transport and release properties and Amphotericin B, alone or in synergism with milk EVs, can exert a therapeutic function. By way of example, such applications include, but are not limited to, the treatment of localized fungal infections via topical application or the treatment of systemic fungal infections via oral administration of Amphotericin loaded milk EVs.

Milk EVs have generally been viewed in the scientific literature as providing a drug carrier for either local applications or for obtaining previously found unsuitable products for oral drug delivery. When experimenting with milk EV preparations according to the present invention, it was surprisingly found that milk EV preparations of the invention can be used to effect extremely efficient transport across the respiratory epithelium and are able to generate high levels of systemic exposure in several tissues, for example liver, kidney, heart or brain and potentially others.

### DESCRIPTION OF THE FIGURES

FIG. 1: Characterisation of different bovine milk EVs preparation steps by Nanoparticle Tracking Analysis (NTA). Particle size distribution histograms, mean and mode size as well as particle concentrations, were determined by NTA. Data are shown for representative samples of milk serum (FIG. 1.1, a-c), pre-processed milk serum (FIG. 1.2, d-f), and EV preparations after Tangential Flow Filtration (TFF) of pre-processed milk serum using two different membranes: with pores of 750 kDa or with pores of 100 kDa (FIG. 1.3, g-i). Particle concentrations are shown as yields, back-calculated referring to the volume of milk serum. For TFF processed material, particle yields and sizes are shown both, for the pre-processed milk serum and retentate (the fluid withheld by the filtering membrane) (h and i graphs). Samples were measured in triplicates; error bars represent standard deviations from the three technical replicates.

FIG. 2: Particle to protein ratios of different bovine milk EV preparation steps. Particle to protein ratios were determined by measuring particle numbers via NTA and total protein concentration using Bradford reagent. Results are expressed as mean of n=3 experiments. (a) Three different samples (batch 1-3) of pre-processed milk serum, (b) two examples of intermediates of TFF processing: Pre-processed milk serum was diluted 1:1 (“input” bar), threefold concentrated and three times dialysed (“retentate” bar) using a membrane of either a 750 kDa or a 100 kDa pores dimension.

FIG. 3: Size exclusion chromatography characterisation of different bovine milk EV preparation steps. Samples were injected onto a Superdex S200 column and eluted at 0.8 ml/min at 4° C. in PBS pH7.4 (Example 14). Chromatograms registered at 280 nm are shown in FIG. 3.1 for milk serum (a), pre-processed milk serum (b), and EV preparations after TFF processing (c). The chromatogram of milk serum is shown as an overlay of the samples before (grey Example 1) and after (black, Example 2) filtration. Panel (b) shows two independent batches of pre-processed milk serum. Panel (c) shows milk EV preparations after TFF processing over a 750 kDa (left) or 100 kDa (right) pore dimensions membranes. The black rectangle highlights the EV peak. FIG. 3.2 panel (d) shows two representative chromatograms of final milk EV preparations following EV isolation according to the method of the present invention using TFF through a 750 kDa pore dimensions membrane (Example 5).

FIG. 4: Relative depletion of protein contaminants during pre-processing (100 kDa molecular weight cut off (MWCO)) of bovine milk EVs. The relative depletion of protein contaminants, as determined by analytical size exclusion chromatography on a Superdex S200 column, was determined by comparing area (black) and height (grey) of peaks 1-6 of milk serum (Example 1) and pre-processed milk serum (Example 4). FIG. 4.1 panel (a) shows the chromatograms registered at 280 nm with peak assignment. Panel (b) shows the peak areas and hights normalised to the EV peak (peak 1). The molecular weight was determined from an independent calibration run with a size exclusion standard. The % depletion of the main contaminants are shown as a function of calculated molecular weight in FIG. 4.2 panel (c).

FIG. 5: Characterisation of different bovine milk EV preparations by Western blotting. Western blot analysis of (a) CD9, (b) tsg101 and (c) MFGE8. HEK Cell lysate (CL) served as a positive control. Bovine milk EVs prepared via different methods are shown: Via Ultrafiltration using a 100 kDa pore dimension membrane followed by Size Exclusion Chromatography (UF SEC), via Tangential Flow Filtration using a 750 kDa pore dimension membrane (TFF, Example 5) and Ultracentrifugation (UC purified milk EVs, Example 12).

FIG. 6: Typical bovine EV preparation by method of present invention. Characteristics of bovine milk EVs processed via Tangential Flow Filtration (TFF) using a 750 kDa pore dimension membrane and the corresponding pre-processed milk serum TFF starting material: (a) SEC chromatogram registered at 280 nm absorption (b) particle size distribution (measured by NTA, histogram represents one out of three measurements), (c) particle to protein ratio, based on particle numbers measured by NTA and total protein measurements by Bradford, n=3, (d) particles per ml milk serum and (e) particle size measured by NTA, n=3. The batch shown was processed by 20 rounds of 4× concentration and dialysis by filling back up to the original volume with PBS pH 7.4 (Example 5).

FIG. 7: Cell uptake of bovine milk EVs into human lung adenocarcinoma cells (A459). TMR/Cy5 labeled EVs obtained in Example 17 were added to A459 cells and cellular uptake was analysed by fluorescence microscopy as in Example 17. Representative images obtained by widefield imaging on a 40×0.95 NA objective are shown in panel (a). BW images: DAPI channel for nuclear staining, TMR channel for EV detection, Cy5 channel for EV detection, and TMR-Cy5 FRET channel; brightfield image in greyscale. Quantification of dose-dependent EV uptake at 6 h in panel (b) for TMR/Cy5 labelled bovine milk EVs stored at either 4° C. or −80° C. Individual curves represent independent biological replicates, whereas 5 individual images were taken per well. Error bars depict standard deviations over the individual images.

FIG. 8: Preparation of goat milk EVs. Characteristics of the following goat milk EV preparation intermediates are shown: Goat milk serum, pre-processed goat milk serum and TFF 750 kDa pore dimension membrane purified goat milk EVs, processed by 20 rounds of 4× concentration and dialysis by filling back up to the original volume with PBS pH 7.4). Samples were characterized in terms of (a) particle size distribution (measured by NTA, histogram shown for one out of three measurements), (b, c) particles per ml milk serum and particle size, both measured by NTA (n=3), and particle to protein ratio, based on particle numbers measured by NTA and total protein measurements by Bradford (n=3).

FIG. 9: Characterisation of goat milk EVs by analytical size exclusion chromatography. SEC chromatograms registered at 280 nm absorbance are shown for (a) goat milk serum, (b) pre-processed goat milk serum and (c) TFF 750 kDa pores dimension membrane processed goat milk EVs. TFF purification was performed by 20 rounds of 4× concentration and dialysis by filling back up to the original volume with PBS pH 7.4.

FIG. 10: Typical goat milk EV preparation by the method of the present invention. Characteristics of goat milk EVs processed via Tangential Flow Filtration using a 750 kDa pores dimension membrane and the corresponding pre-processed milk serum starting material: (a) SEC chromatogram (recorded at an absorbance of 280 nm), (b) particle size distribution (measured by NTA, histogram represents one out of three measurements), (c) particle to protein ratio, based on particle numbers measured by NTA and total protein measurements by Bradford, n=3), (d) particles per ml milk serum and (e) particle size measured by NTA, n=3. GW1PPEK1 was processed by 20 rounds of 4× concentration and dialysis by filling back up to the original volume with PBS pH 7.4.

FIG. 11: Cell uptake of goat milk EVs into human lung adenocarcinoma cells (A459). Goat milk EVs obtained in Example 11 were labelled with TMR and Cy5 as described for bovine milk EVs, added to A459 cells and cellular uptake was analysed by fluorescence microscopy as in Example 17. Representative images obtained by widefield imaging on a 40×0.95 NA objective are shown in panel (a). BW images: DAPI channel for nuclear staining, TMR channel for EV detection, Cy5 channel for EV detection, and TMR-Cy5 FRET channel; brightfield image in grayscale. Quantification of dose-dependent EV uptake at 6 h in panel (b) for TMR/Cy5 labelled goat milk EVs. Individual curves represent independent biological replicates, whereas 5 individual images were taken per well. Error bars depict standard deviations over the individual images.

FIG. 12: Characteristics of bovine milk EVs processed via Ultracentrifugation. (a) SEC chromatogram at 280 nm absorbance), (b) particle size distribution (measured by NTA, histogram shown for one out of three measurements), (c) particle to protein ratio, based on particle numbers measured by NTA and total protein measurements by Bradford, n=3), (d) particles per ml milk serum and (e) particle size measured by NTA, n=3.

FIG. 13: Characterisation of bovine milk derived EVs by TEM with negative staining. Bovine milk EVs of Example 5 were analysed by TEM with negative staining and are shown at 40,000× (a) and 80,000× (b) magnification.

FIG. 14: Quantification of Amphotericin B using high-performance liquid chromatography. RP-HPLC chromatogram of Amphotericin B (50 μl of a 2.5 M solution) with isocratic elution (Acetonitrile/20 mM EDTA (55%/45% vol/vol, 1 ml/min) on a C18 column and 405 nm detection (a) and standard curve for different Amphotericin B (AmB) concentrations derived from the peak area (b).

FIG. 15: Concentration dependence of AmB loading into goat milk EVs. Goat milk EVs at 1 nM were incubated with increasing concentrations of AmB and the concentration of loaded AmB was quantified by RP-HPLC (a). AmB at 2 μM was incubated with increasing concentrations of goat milk EVs and the concentration of loaded AmB was quantified by RP-HPLC (b).

FIG. 16: Co-fractionation of AmB with EVs from bovine and goat milk by size exclusion chromatography. EVs from bovine FIG. 16.1 (a) and goat milk FIG. 16.2 (b) at 1 μM were loaded with Amphotericin B at 2 μM by co-incubation at room temperature for 6 h and the samples were analysed by size exclusion chromatography with UV-VIS detection at 406 nm before and after washing by ultrafiltration. Chromatograms of free AmB and free milk EVs are shown for comparison.

FIG. 17: Uptake of goat milk EVs into A549 cells with and without AmB loading. Goat milk EVs were labelled with TMR-NHS and Cy5 NHS, loaded with AmB and incubated with A549 cells for 6 h. Uptake was then analysed by widefield fluorescence imaging and is shown for AmB loaded versus untreated TMR/Cy5 labelled goat EVs as number of Cy5 positive vesicles per 1000 pixel of cell area. Error bars represent standard deviations over 25 fields of view, data for three independent wells are shown. FIG. 17.1 (a). Exemplary images are shown in FIG. 17.2 (b).

FIG. 18: Susceptibility of Candida albicans to milk EVs loaded with AmpB. The activity of AmpB (unformulated amphotericin B), BMEV (bovine milk EVs), BMEV-AmpB (AmpB loaded Bovine milk EVs), GMEV (goat milk EVs) and GMEV-AmpB (AmpB loaded goat EVs) as prepared in Example 26, was evaluated by measuring the MIC value as described in example 26. A typical plate image (a) and quantitative data (b) are represented.

FIG. 19: characterization of milk EV obtained by different isolation processes (membranes of different pore sizes) using SEC chromatography. Milk EVs obtained using the process of the invention have been isolated using membranes of different exclusion cut-off limits. A: 100 kDa; B: 500 kDa and C: 750 kDa as described in example 29. The samples were then analyzed for their purity using analytical SEC chromatography as described in example 14.

FIG. 20: stability of preparations with different particle concentration upon storage at 4° C. (A) and after freezing at −80° C. and thawing (B). Milk EVs, obtained using the process of the invention, have been isolated as described in example 5 and diluted to the different theoretical particle concentrations from 10\hat{ }13/mL to 10\hat{ }10/mL. The solutions containing the different particle concentrations were then either kept at 4° C. for 3 days or snap frozen at −80° C. and thawed after 24 h before re-measuring the particle concentrations and size distributions with NTA as described in example 13.

FIG. 21: particle concentration (a) and particle size (b) of milk EV after reconstitution of lyophilized (Lyo) or spray-dried (Spray) milk EV preparations. Milk EVs isolated according to this invention and subjected to lyophilization or spray-drying as described in examples 30 and 31, respectively, were reconstituted in water and characterized according to their particle concentrations and size distributions in comparison to the reference sample before drying using NTA measurements as described in example 13. Before measurement the reconstituted samples were optionally filtered using a 0.2 μm syringe filter (fil) or measured as is (non fil).

FIG. 22: IR spectra of milk EV preparations. A: liquid preparation before drying; B: after lyophilization and C: after spray-drying. Milk EVs, isolated according to this invention and subjected to lyophilization or spray-drying as described in examples 30 and 31, respectively, were reconstituted in water and their IR spectra were recorded on using ATR-IR measurements on an AlphaII instrument (Bruker) by applying 3 μL of the liquid to the ATR crystal and letting the sample dry before measuring. The spectra are shown in comparison to the liquid reference before drying.

FIG. 23: IR spectra of milk EV preparations before (A) and after (B) heating (95° C.). The reference sample, before drying, from examples 30 and 31 was subjected to heat stress by heating it to 95° C. on a water bath before recording the IR spectra as described in the above caption of FIG. 22.

### DETAILED DESCRIPTION OF THE INVENTION

When considering large scale purification of milk-derived EVs, several aspects are important; among others, these are: type of starting material, concentration of EVs obtainable (enrichment) and purity of the final product (i.e. how many other non-EV components are present in a typical preparation). The latter is particularly important since contaminating factors may give rise to unwanted side effects and/or interfere with further derivatization of EVs, such as loading with cargoes etc., and/or may complicate the interpretation of data which are generated by preparations of lower quality.

As previously stated, the purification method commonly utilized in the current state of the art for obtaining milk EV preparations is essentially based on several centrifugation steps, at least one of them but commonly two, requiring ultracentrifugation (i.e. >50,000×g), that leads the EVs to form pellets, and thus separates them from the liquid and the contaminating smaller/lower molecular weight components.

The technical draw-backs of such ultracentrifugation process are numerous:
ultracentrifugation is inherently difficult to scale-up and installations are expensive;
ultracentrifugation leads to co-pelletisation of cell debris, as well as protein and oligonucleotide aggregates, such non-EV components often lead to gross-overestimation of EV content;
ultracentrifugation leads to a distortion/modification of EVs (change of size, fusion of vesicles), potentially reducing the biological activity of the preparations containing them.

On the other hand, ultracentrifugation provides a very efficient small-scale methodology, commonly employed in biochemistry to “condense” and extract a material from a liquid based on its specific density (molecular weight), also when scarcely present, and simultaneously remove even a large excess of lower and/or higher density impurities (with lower/higher molecular weight). Applied to EVs isolation, the currently generally accepted method for the preparation of EVs according to the prior art is based on, as first step, pelletising whole cells, organelles lipid aggregates, and larger structures using a lower-speed centrifugation. Such pre-purification step, removing the larger impurities, is followed by an ultracentrifugation step, for example 100,000×g for 90 minutes, to obtain the desired EVs that can also be resuspended and re-ultracentrifuged for higher purity. Some researchers also describe a coagulation step involving the milk proteins, to be carried out before the ultracentrifugation.

Such pre-purification step is for example described, by Wolf et al., 2015, to be 13,200×g for 30 mins, when starting from skim milk, while Izumi et al use 2×1,200×g for 10 mins (Izumi et al., 2015), when starting from raw milk. Other and largely similar values can be found in the field. For example, Gao et al. describe a two-step pre-purification process when purifying EVs from Yak milk, employing first 8,000×g for 30 mins, followed by 13,000×g in order to remove heavier structures (Gao et al., 2019; Gao et al., 2021a; Gao et al., 2021b).

Generally, such removal of larger impurities is followed by an ultracentrifugation step, which according to Wolf et al., 2015, comprises 100,000×g for 90 minutes to obtain a pellet containing the desired EVs, which is then re-suspended and the material re-pelletised under the same conditions. Others (e.g. Izumi et al., 2015) employ for example 100,000×g for 90 minutes and discard the resulting pellet in order to then obtain material from the remaining supernatant by using a higher g force still, of 120,000×g for 90 mins, which is then considered of sufficient quality to be used directly without further washings.

To further assist EV isolation by ultracentrifugation, Gao et al. (Gao et al., 2019; Gao et al., 2021a; Gao et al., 2021b) also describe a process that foresees the coagulation of milk proteins by rennet treatment, prior to subsequently isolating EVs using ultracentrifugation with the commonly known parameters (initial pelletisation at 120,000×g for 90 mins, and washing followed by re-pellitisation using the same parameters).

When considering alternative isolation methods, besides the ultracentrifugation, one candidate methodology is size-based separation based on size exclusion chromatography. Blans et al., (Blans et al., 2017) describe such a methodology in some detail. Their methodology uses a packed column with size exclusion resin Sephacryl S 500 to obtain EV fractions, however only after prior centrifugation at 20,000×g. Furthermore, the methodology described by the authors is only exemplified at small scale (ml processing volumes) and the authors themselves acknowledge that the size exclusion step is used to separate remaining casein and whey proteins from vesicle material after the centrifugation steps either at 340,000×g or at 20,000×g. Therefore, one specific challenge of milk as a starting material is the high-abundance of lipids, fatty acids, and lipoproteins, which in the current state of the art is usually pre-cleared by centrifugation as it would otherwise lead to fouling of size exclusion resin or separation membranes. For this reason, in the current art, these methods have rather been used as supplementary purification methods for milk EV separation after initial (ultra) centrifugation.

It is also important to mention that size exclusion processes with certain resin types (e.g., Sepharose CL-2B), sometimes resulted in EV preparations having modified physicochemical and chemical properties, such as shift to smaller sized particles, modified surface composition, lack of biological activity, etc. Unfortunately, it is still unclear to which parameters of size exclusion resin these detrimental effects are connected to and, for this reason, the size exclusion-based separation processes are, therefore, less preferred.

Membrane based separation has successfully been applied to obtain EV preparations derived from less complex sources compared to milk. In fact, in the case of using milk as raw material for their isolation, specific attention has to be drawn to the other colloidal structures which are present in this natural fluid and are in the same size range as EVs, like milk fat globules (dimension range: 0.1-15 μm) and casein micelles (dimension range: 100-200 nm). In addition, milk contains a large excess of non-EV associated proteins with a wide size distribution and a tendency of coagulation at concentrations above the 10 mg/ml range. Because of these complicating factors, it is commonly known that the use of membrane filtrations, when starting form milk as EVs source, is inconvenient and can lead to the occurrence of artefacts and membrane fouling.

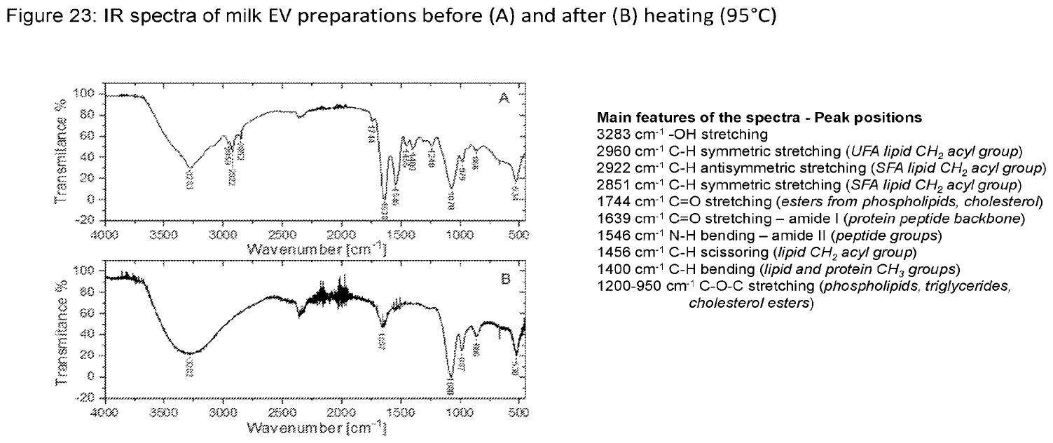

Even so, and contrary to the teaching of the prior art, it was surprisingly found that the process according to the present invention, based on membrane filtration, can successfully be employed for the efficient isolation of highly pure EVs from milk or milk derivatives when preceded by both a pre-purification step of intentional protein-coagulation and a clarification step over an inert filtration bed. Even further, it was demonstrated that the pre-purification step of protein-coagulation, is enhanced when associated by a thermal treatment (pasteurization step), which can increase the microbiological stability of the milk serum after coagulation but, surprisingly, does not affect the stability of the EVs (see experimental section, FIG. 23).

Based on these premises, the present invention is related to a process for isolation of milk-derived EVs and their use as drug carriers, or preparations with intrinsic biological activity, useful for a range of applications comprising, but not limited to, pharmaceutical, veterinary, cosmetic and/or nutraceutical applications or as additives for human or animal diets.

The general term “milk”, if not otherwise specified, is used to define the product made by all mammalian species as is commonly treated in the dairy industry. Examples of milk types comprise, but are not limited to, bovine milk, bovine colostrum, human milk, human colostrum, sheep milk, goat milk, buffalo milk, donkey milk, or camel milk including their respective colostrum forms.

According to the present invention, a process for isolation of purified milk EVs is provided, said process being characterized in that it comprises at least one milk protein coagulation step, at least one thermal treatment shortly after the coagulation step, at least one clarification step and at least one membrane based ultrafiltration and concentration step, providing at least one single homogenous fraction with uniform characteristics, such as in terms of bioanalytical parameters, such as particle concentration, protein-to-particle ratio, chromatographic purity, particle size distribution, etc., Particularly, according to the present invention, the process for isolation of purified milk EVs of the present invention, provides one single homogenous fraction of purified milk EVs with uniform characteristics.

The process for isolation of purified EVs according to the present invention comprises the following steps:
a) providing a sample of milk;
b) optionally clarifying said sample by means of traditionally known filtration or decantation processes, to obtain cleared milk;
c) subjecting said milk or said cleared milk to a treatment with an enzyme mix, suitable for coagulating milk proteins at a pre-defined temperature and time, to obtain a liquid, containing coagulated proteins;
c′) performing a thermal treatment on said liquid containing coagulated proteins;
d) separating said coagulated protein from their supernatant by performing one or more filtrations of said liquid, with an inert layer of a filtration aid, to obtain a clear solution;
e) subjecting said clear solution to at least one ultrafiltration and concentration steps, using membranes of defined pore-sizes and ionic strength and adjusted water or buffer as dialysis medium, in order to obtain an EV concentrated preparation;
f) optionally treating said concentrated EV preparation with a stabilizing agent, such as potassium sorbate, and, concomitantly, adjusting the pH obtaining a stabilized EV concentrated preparation;
g) subjecting said EV concentrated preparation or said stabilized EV concentrated preparation, to a second filtration/clarification step in order to obtain a clarified essentially pure milk EV isolate;
h) optionally subjecting said clarified essentially pure milk EVs isolate to a sterile filtration, using standard sterile filters of typical pore sizes below 1 μm, preferably between 0.45 μm and 0.2 μm, to obtain a clarified essentially pure milk EV sterile isolate.

Optionally, said clarified essentially pure milk EV isolate obtained from step (g) or said clarified essentially pure milk EV sterile isolate obtained from said step (h) may be stored at 4° C. until further use or frozen at temperatures between −1° C. to −80° C. or, more preferably, snap frozen in liquid nitrogen and stored at temperatures between-20 and −80° C., or lyophilized, in absence or presence of common cryopreservatives, such as, but not limited to, mannitol, sucrose, trehalose or proteins such as BSA or casein, to obtain a frozen or lyophilized EVs preparation.

In another embodiment of the present invention, it was found that the EV isolate obtained from the process of the invention is suitable for being conserved by freezing with minimal losses of EV particles upon thawing. This type of procedure, i.e. the freezing of essentially pure EV isolate, requires a starting material containing a high particle concentration, within the order of 10\hat{ }12 particles per ml, more preferably 10\hat{ }13 and above. In general, it was found that EV preparations of certain purity and in high concentration, as they are required for precise, controlled chemical modification and loading procedures, tend to show extensive particle losses after freezing/thawing. Therefore, it is one inherent advantage of the process of the invention, the fact that the resulting EV preparations, consisting of one single fraction of controllable and uniformly high particle concentrations, are suitable for a subsequent freezing process and the following storage in the freeze form.

In fact, with respect to the prior art, the process of the invention consents to obtain sufficiently high concentrations of EVs, which allow, from one hand, the storage of the preparations at 4° C. with minimal or no losses of active particles, and, from the other hand, also the preparation of pharmaceutically acceptable dosage forms via drying processes such as by spray-drying and/or freeze-drying.

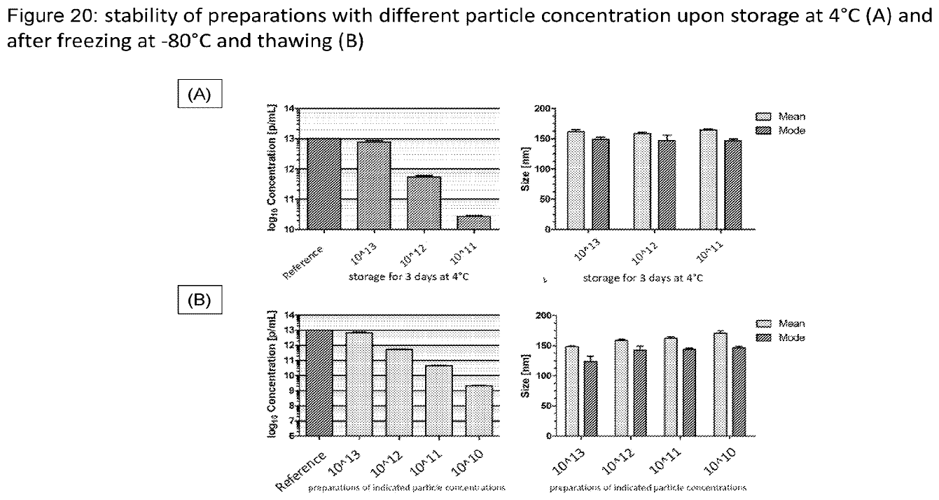

Furthermore, it was surprisingly found that the storability of the milk EV preparations, obtained by the process of the invention, is highly dependent on the particle concentration of the preparation itself. In contrast to protein solutions, which tend to show lower stability with increasing concentration, the milk EV preparations, according to the process of the invention, show increased stability with increasing concentration, as demonstrated by the graphs reported in FIG. 20. Surprisingly, the same trend was observed both for milk EV preparations stored in liquid form at 4° C., as well as for their recovery after freezing at −80° C.

The filtrations according to above indicated steps (d) and (g) of the process according to the invention, can be carried out using any method known in the art, for example, employing disposable or re-useable physical filters comprising a filtration material like a cardboard filter or other filtering cloths or fabric, or using otherwise suitable clarification and filtering methods known in the art.

In the following specification, different embodiments of the present invention will be indicated, as part of the present invention. The information provided herein, along with the specific details according to the examples, are hereby reported for clarity purposes and should not be understood as limitations of the invention nor should unnecessary limitations be concluded from these exemplary embodiments and the details contained in the examples.

Please note that, in an embodiment of the present invention, various pre-processed forms of milk or milk derivatives may be used as starting materials instead of the plain milk sample. For example, but not limited to, milk powder, skimmed milk, heat-treated milk, pasteurized milk, or colostrum, provided that these derivatives contain “intact” EVs, i.e. EVs having the potential to exert their biological functions.

When milk powder or colostrum powder are provided, the process of the present invention further comprises a re-hydration step, in which said powders or granulates are dispersed in water to give a uniform suspension. This is usually achieved by combining the powders or granules with water and agitating the mixture to produce a homogenous suspension. All known re-hydration and suspension methods will be suitable for said additional step, provided that they do not damage the EVs particles.

In another embodiment of the present invention, step (c) is performed by using an enzyme mix containing pepsin and chymosin, which is suitable for casein-based coagulation of the milk proteins. Enzyme mixtures able to achieve this coagulation are, for example, composed by pepsin (in a range from 0 to 90%) and chymosin (in a range from 100 and 10%). Preferred mix are composed by 5% pepsin and 95% chymosin, even more preferred 20% pepsin and 80% chymosin. The enzyme mix can be used in a quantity and for times well known by the skilled in the art, for example about 40 mg of enzyme mix or about 50 International Milk Clotting

Units (IMCU) per litre of milk. The reaction time will depend from the enzyme mix quantity and may range from few minutes to several days. In a preferred embodiment of the invention, temperatures in the range of 25-37° C. are used to carry out the reaction. The pH is either adjusted to a slightly acidic value, between 5 and 7, using standard methods and ingredients known in the art, or left unadjusted at the initial value of the starting material.

The most preferred enzyme mixture to be used according to the invention is rennet form bovine origin, containing 5% of pepsin and 95% of chymosin. The possibility of employing such enzyme mixtures can be considered one of the many advantages of the present invention with respect to the prior art which describes in contrast, for example, the use of acid solutions or complexing agents like EDTA. The rennet, in fact, is currently used for the production of different types of cheeses and, therefore, can be considered totally safe for its use in the isolation process of EVs to be further used for human or animal administration. However, according to another embodiment of the present invention, also other enzyme mixtures, which are derived by fermentation, including pure chymosin, may be used.

In the present invention, after said step (c) a further step (c′) is envisaged, in order, as previously clearly explained, to improve the microbiological characteristics of the product. In particular, a thermal treatment, also called pasteurization, is performed. Said thermal treatment consists in the heating of the liquid containing coagulated proteins obtained in step (c) to a temperature above the ambient temperature and below 95° C. for a short period of time (i.e. in the range of the tents of minutes). Preferably, said liquid containing coagulated proteins obtained in step (c), is heated to 45-65° C., preferably between 53-56° C., for at least 10-20 minutes.

As previously reported, the thermal treatment (c′) does not impair the activity nor the integrity of the EVs, which are present in the liquid obtained in step (c).

Thereafter, the mixture is cooled and subjected to one or more filtrations, according to step (d). In a preferred embodiment of the present invention, step (d) contemplates a first coarse filtration to remove most of the coagulated protein, followed by depth filtration using a filtration aid like diatomaceous earth or similar filtration aids, as are commercially available and routinely employed in depth filtration as filter layers. The clarified material obtained at the end of step (d) has a refractive index of 3-5° Brix.

The clarified and de-caseinated liquid obtained at the end of step (d) is then subjected to a series of at least one concentration and dialysis step according to step (e). In a preferred embodiment a first pre-concentration step is carried out, followed by dialysis to efficiently remove the remaining contaminating proteins and other smaller components, before a final concentration step is performed in order to reach the desired final concentration of the milk EVs.

Membranes of different pore sizes have been tested for the concentration and dialysis steps. Generally, most of the contaminating proteins will be removed by membranes having a size exclusion cut-off limit above 70 kDa, more appropriately having a cut-off close to or above 100 kDa, even more appropriately, having a cut-off of close to or above 300 kDa and more preferably a membrane with cut-off of 750 kDa. Particularly, the use of a membrane with a molecular weight cut-off at or close to 750 kDa in at least one ultrafiltration step following casein coagulation, allows to eliminate virtually all contaminating non-vesicular proteins (as measured by analytical SEC chromatography on e.g., a Sephadex S-200 30/10 column). The choice of such membrane is not a priori obvious to those skilled in the art since on one hand contaminating proteins typically have molecular weights much smaller than 300 kDa and on the other hand EVs have been shown to pass essentially unhindered through membranes having 0.22 μm pore sizes or inert filtration aids, able to remove soluble aggregates thus suggesting that larger membrane sizes might lead to significant losses of EVs while not improving protein elimination. Also, for non-milk EVs the use of ultrafiltration membranes with molecular weight cut-offs significantly larger than 100 kDa, such as 300 kDa and above has been associated with EV losses. Furthermore, membranes with molecular weight cut-offs of 750 kDa are not usually commonly available, especially not in module sizes, allowing a larger scale industrial purification, which usually are performed with spiral filter modules inserted in metal housings. Such filters are commonly available with cut-offs from 3 kDA, 10 kDa, 30 kDa, 100 kDa and 500 kDa.

Please note that, for the isolation method of the invention, the fraction to be recovered (i.e. the one containing the EVs) is the one remaining above the filter, not the fraction passing through. In a preferred embodiment of the present invention, in order to achieve the desired concentration and purity, a first pre-concentration is carried out, during which the material is concentrated several folds, typically 2-6-fold with an appropriate membrane. This first pre-concentration is followed by another filtration, in dialysis mode, and at least 5-10 volumes are exchanged. It has been found that for this dialysis the ion strength of the water or aqueous buffer has to be controlled in order to prevent aggregation of remaining macromolecules and the solutions from becoming turbid. The inclusion of sodium chloride in concentrations in the range between 0.5 and 1.5%, more preferably, 0.8-1% have been found to be sufficient for this purpose. For those skilled in the art, other solutions will be evident such as change of the specific salt form or change to one of the numerous buffer systems used in biochemical preparations, such as but not limited to, buffers based on phosphate, citrate, carbonate, Tris, HEPES, and others.

At the end of the dialysis, the EV preparation can be concentrated further to the desired end-concentration of EVs. This end concentration can be determined by methods established in the art, such as Nano-Particle Tracking analysis. Typically, values for achieved particle numbers with the method according to the invention are in the range of 10\hat{ }12-10\hat{ }13 particles/ml with typical values of particles/mg of total protein being also in the range of 10\hat{ }12-10\hat{ }14 particles/mg protein.

As the final step of the preparation method, the material obtained from step (e) is further clarified in step (g), for example by using diatomaceous earth like in step (d).

Optionally such a filtration step might be required also after the first ultrafiltration and concentration steps of the pre-processing or during ultrafiltration if, for example, the material to be processed is particularly rich in fat, depending on the exact characteristics of and the species from which the starting material is derived. Such filtration steps also serve to remove early on soluble protein aggregates, derived e.g. from the sheer stresses the material is subjected to during dialysis and which otherwise can catalyse or induce further protein aggregation. Such aggregates can further lead to membrane fouling during subsequent dialysis and concentration steps, or give rise to precipitation and loss of EV material after prolonged storage in liquid or after a freezing process or even after reconstitution from the lyophilized (freeze-dried) or spray-dried forms.

These clarification filtration steps can be useful to obtain an aggregate-free, well soluble and stable EV preparation.

Optionally, in a further embodiment of the invention, the preparation can be subjected to a further filtration, which reduces microbial contamination or to an actual sterile filtration (h). The skilled in the art is perfectly capable of choosing a suitable method for this step, for example, the use of standard sterile filters with pore sizes below 1 μm.

Moreover, in another embodiment, different methods can be applied to the preparation in order to improve its stability and shelf life. By way of examples, stabilizers can be added to the final product and/or the EV extracts can be frozen, with or without different cryoprotectants, lyophilized and/or spray-dried.

The present invention therefore also relates to EV preparations, obtained via the process above, which satisfy several, exceptionally high, quality criteria. Such tight quality standards are important for using natural isolates, like EV in pharmaceutical preparations or as part of finished dosage forms in pharmaceutical products. EV preparations according to the current invention are essentially pure EV preparations and exhibit the following analytical characteristics: high particle concentrations of at least 10\hat{ }12 particles/ml, more preferably at least 10\hat{ }13 particles per ml or even more preferably at least 10\hat{ }14 particles as determined by NTA analysis and typically a high particles to protein ratio on the order of 5×10\hat{ }12 particles/mg protein or more preferably at least 10\hat{ }13 particles/mg protein and an EV peak purity of not less than 75% or more preferably at least 80% and more preferably still having an EV peak purity of more than 85% or most preferably having an EV peak purity of more than 90%, as measured by FPLC using an analytical Sephadex-S200 30/10 column at 280 nm detection or equivalent chromatographic set-up.

In another embodiment, the current invention provides a method for preparing pharmaceutically acceptable dosage forms and storage forms. In particular, the process according to the current invention consent to obtain preparations in which the particle concentration is within values allowing an increased stability of the preparation itself, i.e. approximately 10\hat{ }12 particles/ml, more advantageously around 10\hat{ }13 particles/ml, even more advantageously, more than 10\hat{ }13 particles/ml. In addition, the buffer composition may be controlled during the dialysis step in order to obtain preparations of certain profitable characteristics.

For example, it is commonly considered that EV preparations require an accurately controlled, isotonic environment. However, it was surprisingly found that, while this is important during the first cycles of dialysis, when carrying out the process according to the current invention, the later dialysis cycles can be utilized to eliminate the salts and to change into a pH regulating buffer with a buffer strength below isotonic values (e.g. 10 mM Phosphate buffer pH 7.4). While other tonicity regulators, such as glycine or sucrose can be included, it was surprisingly found that such additives are not strictly required for the integrity of the EVs derived from milk, in particular in the case of the preparations deriving from the process of the invention which presents the previously discussed concentration values. This observation is particularly true, especially once the contaminating non-EV associated proteins have been reduced from their original concentration.

Accordingly, the current invention provides the possibility to obtain milk EV preparations which are essentially free from non-pH-active salts, such as NaCl, KCl, Na₂SO₄, etc. which form part of the tonicity regulating properties of physiological buffers, such as PBS (phosphate buffered saline) or other tonicity regulators. Such salt components, or other additives, will greatly be enriched in concentration in the drying process (see a more in depth explanation below) but exhibit a wide range of undesirable properties, due to their physiological activities, anti-nutritional values, bitterness, or the like.

Even highly concentrated milk EV preparations contain a relatively low percentage of dry matter as compared to protein solutions. This is due to the much larger volume the EVs occupy. Typically, essentially pure milk EV preparations according to the present invention, exhibit EV associated dry matter contents of well below 15%, usually below 5%, and more typically below 2%.

If the milk EVs are prepared in isotonic solutions, such as PBS, which contains about 1% of sodium chloride, the resulting dry powders will contain high amounts of salt. Moreover, during the drying process the salt concentration constantly increases until the mass is dry. This leads to protein denaturation, and can damage the EVs during the drying process. In addition, for certain experimental testing and dosage forms (e.g. in the case of oral delivery), high quantities of salt are undesirable as they affect the organoleptic properties of the material.

As previously mentioned, the process according to the current invention, is able to solve the problem mentioned here below thanks to the fact that, after the first dialysis cycles in an isotonic media and the elimination of the protein contaminants, can after switch to a buffer system which is only pH regulating, and only optionally can contain other additives or excipients, as required.

In a particular embodiment, such final preparations obtained by the process of the present invention, can then be subjected to freeze-drying (also referred as lyophilization) or spray-drying in order to obtain milk EVs in dry form.

Notably, it was surprisingly found that, the milk EV preparations according to the current invention, which contain a suitably high particle numbers and are free from tonicity regulators excipients, can be lyophilized without the need for a snap-freezing passage (a rapid freezing of the sample) before lyophilization.

In a preferred embodiment, the milk EV preparations according to the current invention are subjected to an initial exchange of around 5 volumes of an aqueous solution containing about 1% salt (e.g. NaCl), as described above or other buffer, and then other around 5-10 volumes with a solution containing only a buffering agent (e.g. phosphate buffer, made from potassium phosphate 10 mM, for a pH 7.4) and optionally other excipients. The final preparations can be lyophilized, following the procedures well known by the expert in the field, feeding the lyophilizer with the liquid sample, freezing it in the lyophilizer according to standard industrial practices (e.g. lowering the temperature by ca. 0.1° C. per minute and initiating the vacuum when the product has reached ca. −20° C.). The parameters of the lyophilization can be adjusted by the skilled man as per its experience; keeping in mind the consistent advantage related to the fact that no snap-freezing nor pre-freezing of the material is required.

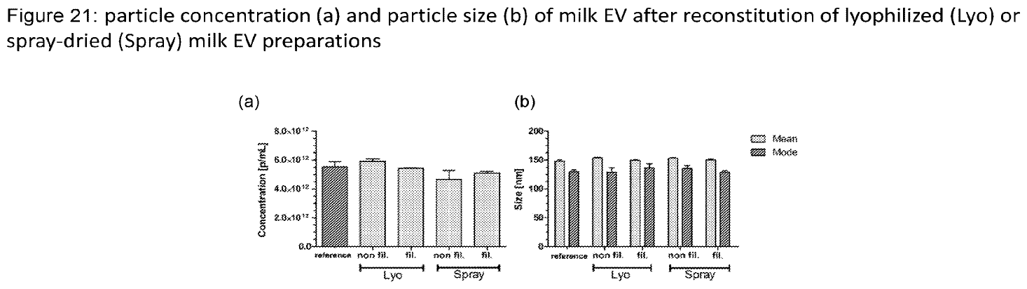

Lyophilized preparations of milk EVs according to the described procedure were surprisingly found not to differ in terms of particle size and particle numbers after reconstitution with respect to the milk EVs which were not subjected to the freeze-drying process (see experimental section and FIG. 21).

In another embodiment of the present invention, the milk EV preparations according to the invention are subjected to a spray-drying process. While lyophilization exposes the preparation to a stress derived from freezing, and the associated effects of water crystal formation, as well known in the field of protein and cell therapeutics, spray-drying is a process generally considered less appropriate for sensitive macromolecules and/or cell derived therapeutics due to the increased thermal stress to which the material is exposed during the drying. In fact, the typically employed drying parameters are an inlet temperature of ca. 150-175° C. and an outlet temperature of ca. 70-85° C. Despite this considerable heat stress imposed on the product, it was surprisingly demonstrated that, also in this case, powders with good reconstitution properties (i.e. practically unchanged particle sizes and concentrations) can be obtained (FIG. 21).

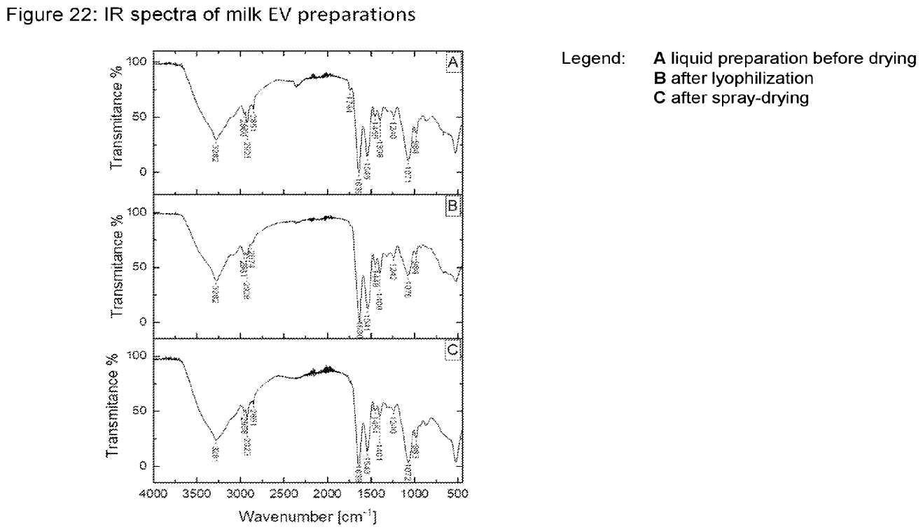

Another proof of the ability of those two drying techniques not to impact on the milk EV structure and function can be found also in FIG. 22: when reconstituted in water, the dried forms of the milk EV preparations according to the present invention did not result in any significant changes in their respective IR spectra.

The present invention also concerns the use of the EV product, isolated from milk or milk derivatives following the method according to the invention, in the pharmaceutical, veterinary, nutraceutical and/or cosmetic fields. In particular, the obtained EVs can be decorated, using the methods known in the art, in order to enhance their uptake and/or target specific organs or tissues, and/or they can be loaded with active principles and, consequently, employed as drug carriers and delivery systems.

In fact, the EV preparations obtained according to the present invention are particularly suitable for use as drug carriers, since they are characterized by a low degree of contaminating proteins otherwise typically arising from co-precipitation with EVs during ultracentrifugation using milk as starting material. Such protein contaminants and co-precipitates are known to interfere with subsequent modification and loading steps of exosomes and/or can give rise to false positives or unwanted side-reactions during, for example, bioconjugation, lipid-insertion or other chemical or physical methods for loading EVs with active drug molecules or pro-drugs.

The terms “active principle” and “drug” can be considered synonyms and they can indicate different types of molecules or compounds able to have an effect on the human or animal body in terms of health improvement or treatment or prevention of syndromes or diseases. Besides the known pharmaceutically active compounds, in the present invention also compounds with cosmetic and/or nutraceutical value can be considered an “active principle”. The person skilled in the art is perfectly able to select the more suitable compound, and the correct way for its loading into the EVs, with respect to the aim for which the EV preparation is envisaged.

In a specific embodiment of the present invention, the EVs derived from milk or milk derivatives following the isolation process of the invention, are loaded with amphotericin B, a well-known compound with antifungal and antiparasitic activities.

As with many other delivery systems, the specific effects obtained with a given compound loaded into an EV, cannot be generalized or predicted upfront but need to be experimentally tested. In fact, many different parameters can generally affect the final result. Among such parameters, for example, the stability of the association of the drug molecule with the proteins, lipids etc. presented on the outer surface of the EVs has to be considered, as well as its ability and efficiency in crossing over the membrane barrier towards the space inside of the EV particle, and, moreover, its ability and efficiency in being set free from the EV during the “delivery phase”. Also if a case-by-case study is therefore needed, no matter whether a specific drug and loading method turns out to be suitable for being delivered with milk EVs, it is important to highlight the fact that the preparations of the EVs of the present invention, due to their purity and high concentration, are exceptionally well suited for the task.

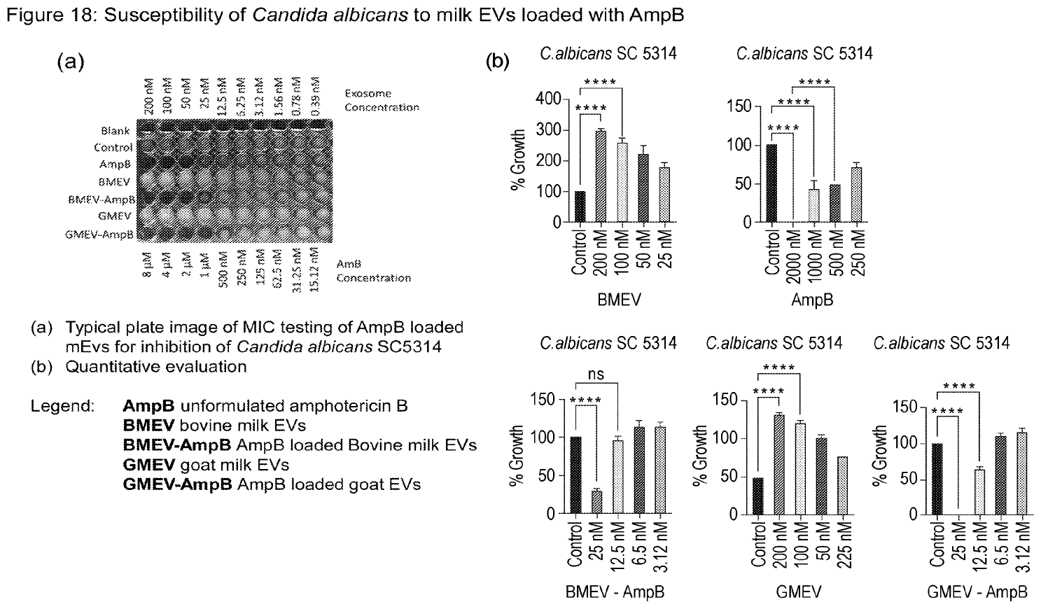

In a particular embodiment of the present invention, amphotericin B was loaded into milk EVs and tested for its MIC (Minimum Inhibitory Concentration) values on several strains of Candida albicans also in comparison to unformulated amphotericin B. These tests showed a roughly 50-fold lower MIC value for amphotericin B when loaded into milk EVs, as compared to unformulated amphotericin B, demonstrating that the delivery system with the milk EVs is very efficient in delivering the drug into the fungal cells (Example 26, FIG. 18).

A further aspect of the present invention is related to the preparations containing the EVs, isolated following the process of the invention, used to administer them, and their loaded drug if present, to a human or animal subject.

In particular, the EVs, eventually decorated and/or loaded with one or more active principles, can be formulated in different preparations for the systemic and/or topic administration. For example, but not limited to, the EVs can be mixed with convenient excipients in order to obtain suitable formulations for the oral and/or parenteral administration, for inhalation, for topic administration on the skin or the mucosae.

Thanks to their intrinsic characteristics, in fact, the EV particles are particularly biocompatible with the biological barriers of the human and animal bodies. Their structure and composition can ease the absorption of the EVs at the cellular membrane level, enhancing, as a consequence, the bioavailability of the loaded drug every time a physical barrier is present. By way of example, the use of EVs as delivery system, can enhance the bioavailability of the conveyed drug along the gastrointestinal tract, the bronchi mucosae and/or the skin, leading to an enhanced concentration of the active principle in the bloodstream and at the target site.

In a preferred embodiment of the present invention, the milk EV preparations have been found to be particularly suitable for inclusion in a formulation for inhalation.

Administration of milk EV preparations, according to the current invention, have surprisingly been found to affect a very efficient systemic exposure after bringing them in contact with the respiratory epithelium of individuals of all ages including adults.

It is therefore surprising and of note that for the efficient systemic delivery mechanism via the respiratory epithelium according to the present invention, no further modification of milk EVs is required and that such systemic exposure following contact with the respiratory epithelium has been found to be several orders of magnitude higher than systemic exposure obtained with equivalent dosages of milk EVs via the oral route. After application of small volumes of milk EV preparations via the nasal route, systemic exposure in several tissues, including brain tissue and liver have been detected in adult mammals.

All the discussed advantages presented by the invention will further be proven in the following experimental part, that is intended not to be limiting with respect to the whole scope of the invention previously reported.

### EXPERIMENTAL SECTION

### Example 1

Fresh cow milk (2.5 L) was collected and left to stand in a graduated cylinder at 10° C. for 22 h, during this time natural creaming occurred. The top layer of the milk (ca. 150 mL) was removed by decanting.

The pH of the skimmed milk was adjusted to pH=6.4 using citric acid. Then the skimmed milk (2 L) was preheated to 35° C. for 15 minutes with a heat exchanger unit connected to a jacketed tank. To this heated solution, rennet of bovine origin, containing 20% pepsin-80% chymosin, was added at a dosage of 50 IMCU (International Milk Clotting Units) per L. The coagulation temperature was maintained at 35° C. for 25 minutes. After which the mixture was heated, as quickly as possible, to 56° C. and at that temperature was held for 10 minutes. The mixture was then quickly cooled to below 10° C. and the liquid milk serum separated from the solid coagulated casein protein mass using a sintered glass funnel of porosity 1 with a filtration cloth inlet. In total about 1.7 L of slightly turbid milk serum was obtained.

The resulting material was characterized with respect to particle numbers and size distribution, as well as total protein content as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| particles/ml | SD | mean | SD | mode | SD | mg/ml | SD | protein | milk serum) |
| 1.14E+12 | 1.29E+11 | 186.5 | 1.6 | 154.8 | 5.8 | 1.55 | 0.04 | 7.32E+11 | 1.14E+12 |

### Example 2

The turbid milk serum (1.5 L) obtained in Example 1 was subjected to clarification by filtering over a filtration aid (Diacel CF/S with 0.1-0.2 Darcy units, medium particle size 13 μm) using a standard sintered glass filter funnel of porosity 3 to obtain a clear yellowish solution with a refractive index of about 6.

### Example 3

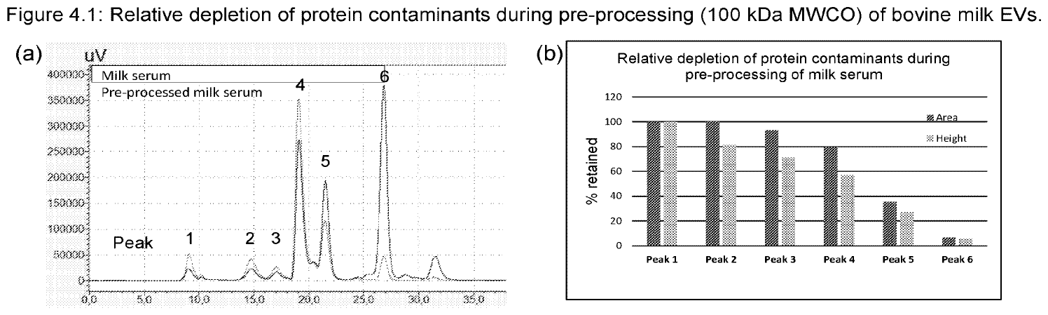

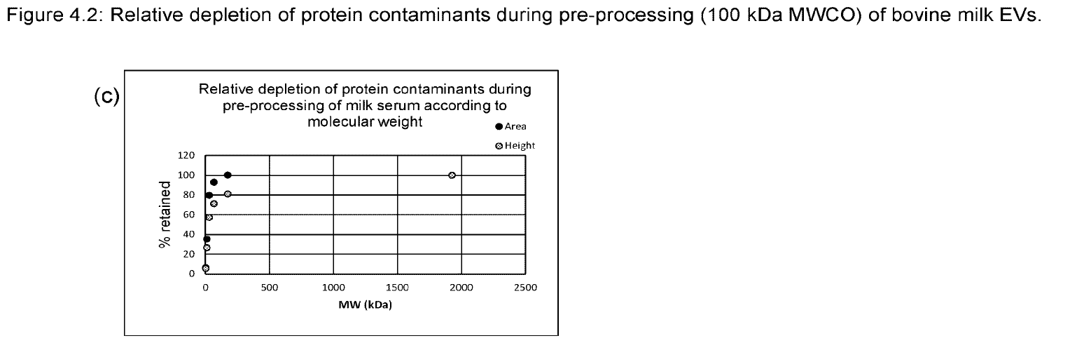

The product from Example 2 (1.5 L) was then processed using a Vivaflow 200 TFF system (Sartorius) with disposable Membrane Cartridges having a MW-cutoff of 100 kDa. The material was first concentrated four times to reach a total volume of ca. 370 mL. Then, four volumes (1.5 L) of a 1% sodium chloride solution were added and the material concentrated again to the previous volume of 370 mL. The relative depletion of protein contaminants with different molecular weights during this pre-processing step was determined by analytical Size Exclusion Chromatography (SEC), as described in Example 14, and is shown in FIG. 4.

### Example 4

The product from Example 3 was clarified following the procedure described in Example 2. After clarification, the sample was sterile filtered over a disposable 0.45 μm filtration unit to obtain ca. 350 mL of a clear opaque solution with a refractive index of 2. The product was then split into different aliquots of 50 mL each and some aliquots were shock frozen for long-term storage.

The resulting material was characterized with respect to particle numbers and size distribution, as well as total protein content, as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 1.80E+12 | 4.97E+11 | 161.1 | 2.3 | 147.0 | 9.6 | 1.14 | 0.14 | 1.57E+12 | 4.49E+11 |

### Example 5

The frozen product from Example 4 (4×50 mL) was slowly thawed over-night and re-clarified with the method described in Example 2. The material was then subjected to a second concentration/dialysis cycle using a hollow-fibre membrane with a 750 kDa molecular weight cut-off (MWCO) (D02-E750-10-N mPES). The material was first concentrated four times to a volume of 50 mL. Then a total of 20 volumes were exchanged by dialysis by repeatedly adding 100 mL of PBS pH 7.4. Finally, the sample was concentrated to obtain ca. 40 mL of purified and concentrated milk EVs. The material was sterile filtered using a 0.2 μM PES syringe filter. The resulting material was characterized with respect to particle numbers and size distribution, as well as total protein content, as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 9.26E+12 | 5.90E+11 | 165.33 | 0.6 | 146.17 | 7.87 | 1.67 | 0.016 | 5.55E+12 | 2.01E+11 |

### Example 6

Fresh cow milk (1,500 L) was collected and partially skimmed by letting it settle in a tank over 24 h.

The skimmed milk (1,300 L) was then preheated to 35° C. for 15 mins. To this heated solution, rennet of bovine origin (containing 5% pepsin-95% chymosin) was added at a dosage of 50 IMCU/L. The coagulation temperature was maintained at 32° C. for 60 minutes. After which the mixture was heated as quickly as possible to 56° C. and at that temperature was held for 15 minutes. The mixture was then let to cool down and the liquid milk serum separated from the solid coagulated casein protein mass using a metal mesh filter.

In total about 1,000 L of slightly turbid milk serum were obtained.

### Example 7

The milk serum (600 L) obtained from Example 6 was clarified using a plate and frame filtration device. The filter press was first assembled with filter sheets (Pall EK filters) and pre-coated by passing a solution of filtration aid (Diacel CF/S, 10 kg) in water. Then, other 5 kg of that filtration aid were added to the milk serum and the serum was passed over the filter. 500 L of clear serum were obtained after filtration. The thus obtained clear serum was then concentrated to 100 L using a 5″ hollow fibre cartridge with a MWCO of 100 kDa. Then, 500 L of a 1% NaCl solution in distilled water were added to the product and the solution was re-concentrated to a final volume of 100 L. This material was then clarified another time using the depth filtration setup and immediately after the filter passed through a sterile filter cartridge (Filtrox, 0.5 μm). Thus, 80 L of clarified pre-concentrated milk EVs were obtained and frozen.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 5.52E+12 | 5.90E+11 | 147.1 | 0.8 | 139.2 | 5.1 | 1.54 | 0.021 | 3.57E+12 | 7.36E+11 |

### Example 8

A sample of 6 L obtained from Example 7 was concentrated sixfold (to 1 L) and subjected to six dialysis/concentration cycles as described in Example 5 using a hollow-fibre membrane with a 750 kDa MWCO (D02-E750-10-N mPES) by addition of 6 volumes (6 L) of PBS (pH 7.4) to obtain 1 L of sterile filtered and purified milk EVs.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 2.41E+13 | 4.40E+11 | 142.4 | 1.7 | 126.0 | 5.3 | 1.0 | 0.021 | 2.41E+13 | 5.13E+11 |

### Example 9

Milk serum (2,000 L) obtained as described as in Example 6 was clarified using a plate and frame filtration device. The filter press was first assembled with filter sheets (Pall EK filters) and pre-coated by passing a solution of filtration aid (Diacel CF/S, 10 kg) in water. Then, other 5 kg of that filtration aid were added to the milk serum and the serum was passed over the filter. 1800 L of clear serum were obtained after filtration. The thus obtained clear serum was initially concentrated to 400 L using a 5″ hollow fibre cartridge with a MWCO of 500 kDa. Then, 2,000 L of a 1% NaCl solution in distilled water were added to the product and the solution was re-concentrated to obtain 100 L. At this point other 500 L of PBS (pH 7.4) were added to the product and the solution was concentrated to ca. 60 L. This material was then clarified another time using the depth filtration setup and immediately after the filter passed through a sterile filter cartridge (Filtrox, 0.5 μm). Thus, 50 L of purified milk EVs were obtained.

### Example 11

This example proceeds like Examples 1 to 5, starting from fresh goat milk (2.5 L) to obtain 20 mL of purified goat milk EVs.

Goat milk was pre-processed following the steps described in Examples 1-4. 150 mL of pre-processed goat milk serum was slowly thawed over-night. The material was then subjected to a second concentration/dialysis cycle using a hollow-fibre membrane with a 750 kDa MWCO (D02-E750-10-N mPES). The material was first concentrated four times to a volume of 38 mL. Then a total of 20 volumes were exchanged by dialysis by repeatedly adding 112 mL of PBS pH 7.4. Finally, the sample was concentrated to obtain ca. 20 mL of purified and concentrated goat milk EVs. The material was sterile filtered using a 0.2 μM PES syringe filter. The resulting material was characterized with respect to particle numbers and size distribution as well as total protein content as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 1.18E+13 | 1.12E+12 | 137.5 | 1.5 | 103.0 | 6.4 | 1.44 | 0.13 | 8.19E+12 | 4.33E+11 |

### Example 12

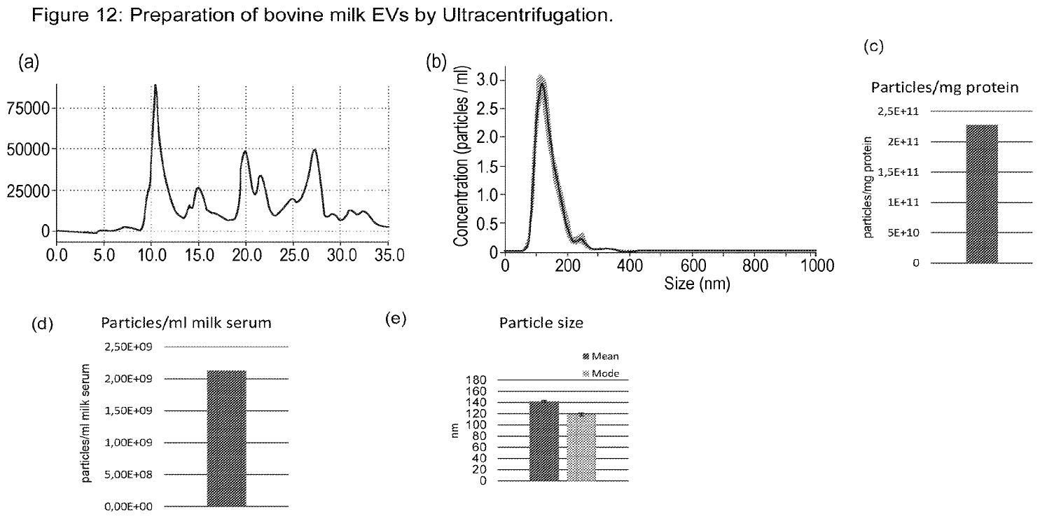

For comparison, a literature-based milk EV isolation protocol based on ultracentrifugation was carried out. The protocol was based on experimental procedures reported in the materials and methods sections of Betker et al., 2019; Agrawal et al., 2017; Munagala et al., 2017. Fresh whole milk was defatted by spinning for 30 min at 4° C. at 13,000 g. Supernatant was passed through a Whatman filter paper. Defatted milk was centrifuged for 60 min at 4° C. at 100,000 g (corresponding to max rcf 31,400 rpm, Sorvall Discovery Ultracentrifuge, T865 fixed angle rotor). The supernatant (not more than 70% of the total volume) was carefully harvested with a glass pipette without disturbing the lower slush layer. EVs were pelleted by spinning the supernatant for 90 min at 4° C. at 135,000 g, corresponding to max rcf=36,400 rpm (FIG. 12). Pellets were washed 3 times with PBS pH 7.4, dissolved in PBS pH 7.4 and sterile filtered using a 0.2 μM PES syringe filter.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 1.14E+11 | 0.20E+11 | 142.8 | 1.6 | 119.0 | 3.0 | 0.287 | n.a. | 2.28E+11 | 2.13E+09 |

### Example 13: Nanoparticle Tracking Analysis (NTA) of EV Preparations

Particle numbers and size distribution were analysed using a NanoSight LM10 instrument (Malvern), configured with a 488 nm laser. Videos were collected and analysed using the NTA 3.1 software.

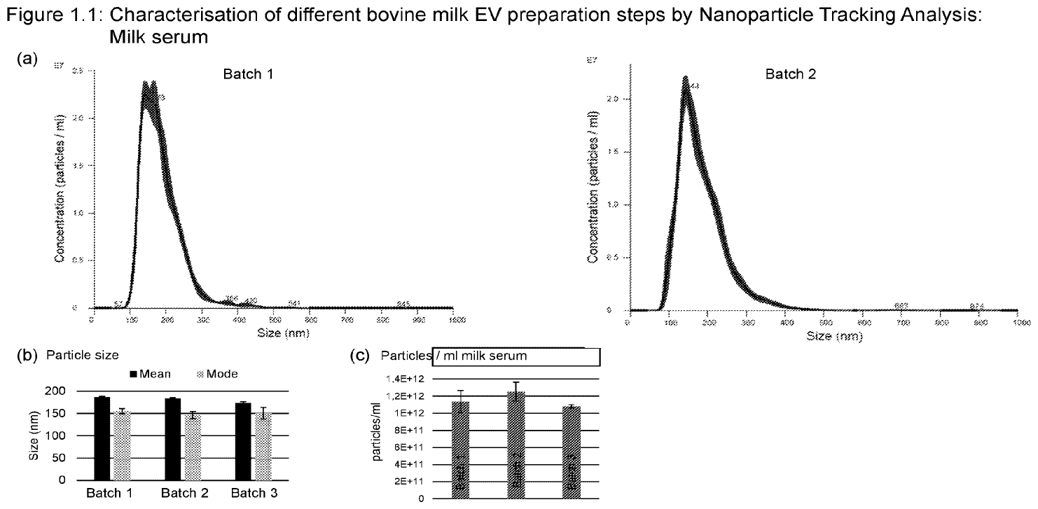

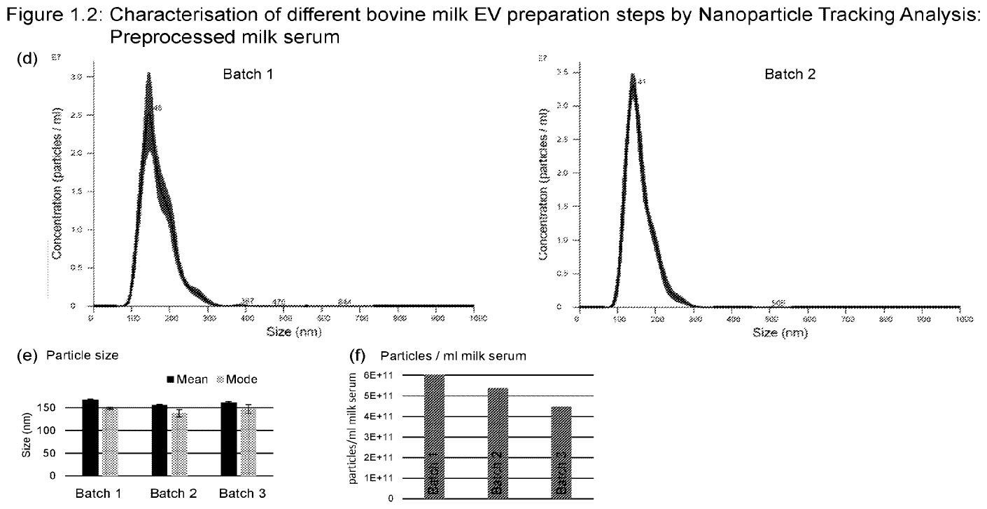

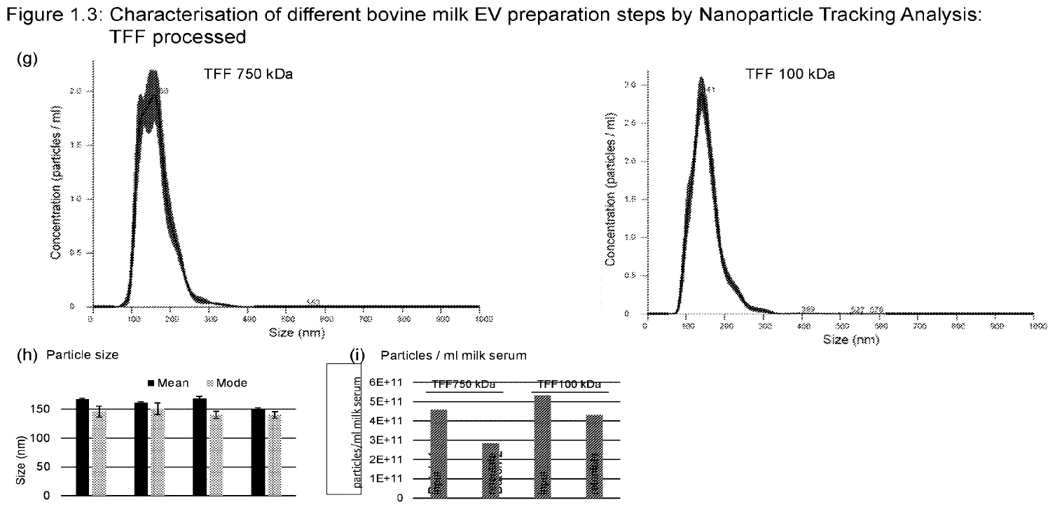

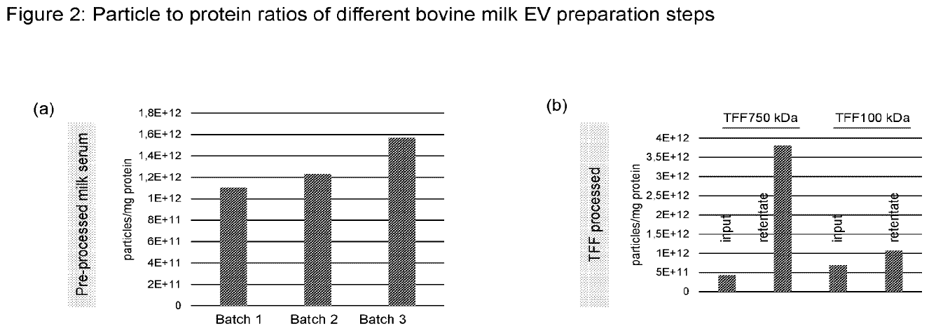

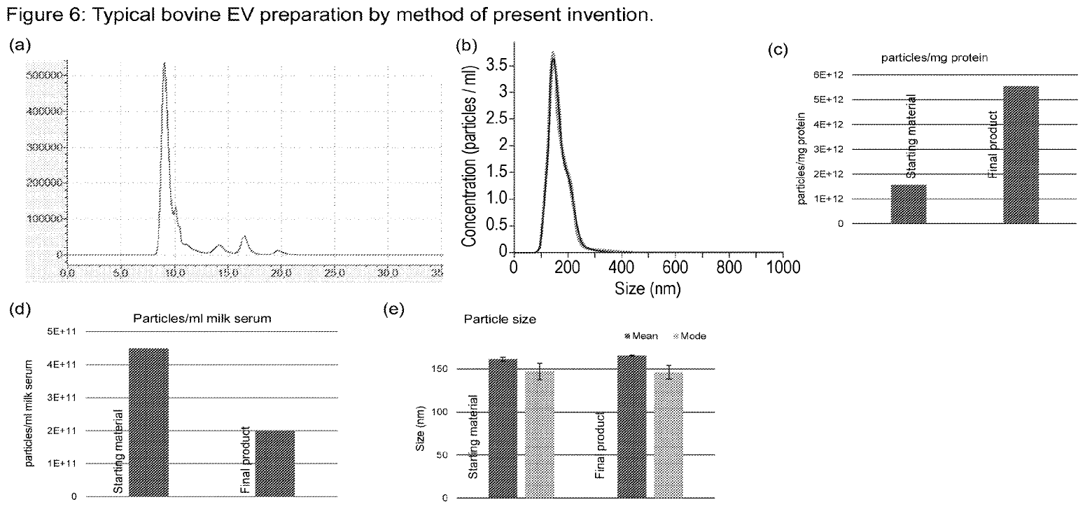

Measurements were performed in triplicates at a controlled temperature of 25° C. Each sample was diluted 1 to 100 in sterile filtered PBS pH 7.4 to 1 mL. Samples were further diluted in order to perform measurements in a range of 80-120 particles/frame. The camera level was kept at 12 during all measurements, and five consecutive 60 seconds recordings were made for each sample. Samples were analysed with a detection threshold of 5. Characterisation of Milk EV preparations by methods of the present invention using NTA is illustrated in FIGS. 1, FIG. 2, 6 (b-e), 8, 10 (b-e), and 12 (b-e).

### Example 14: Size Exclusion Chromatography (SEC) Analysis of EV Preparation

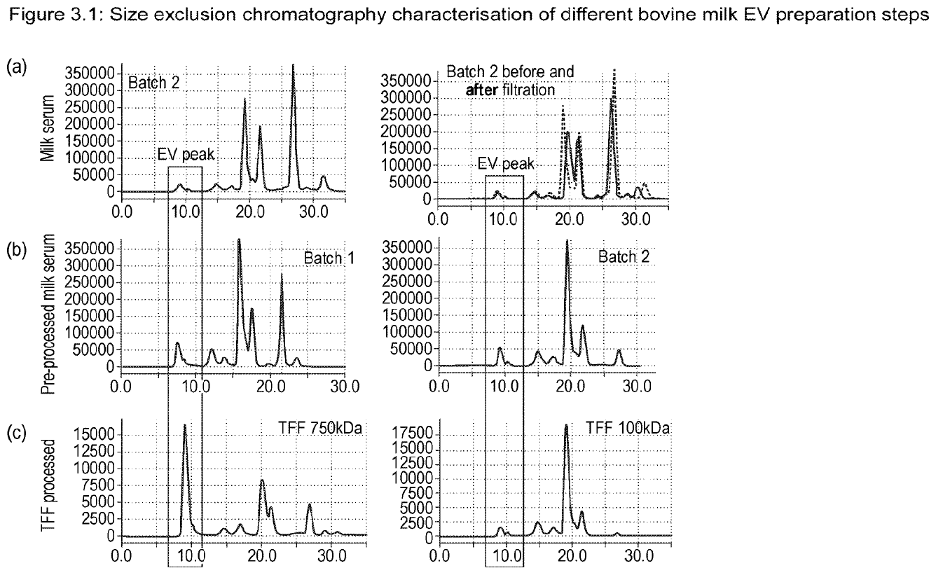

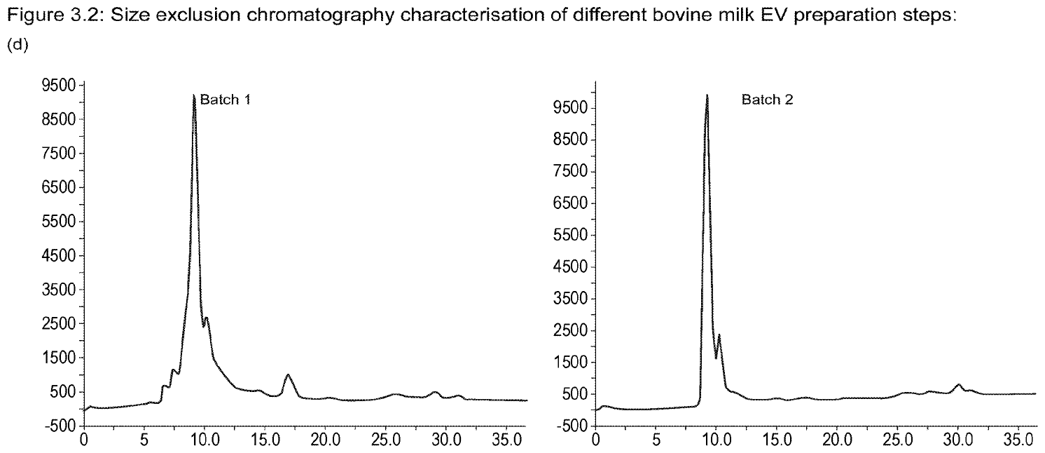

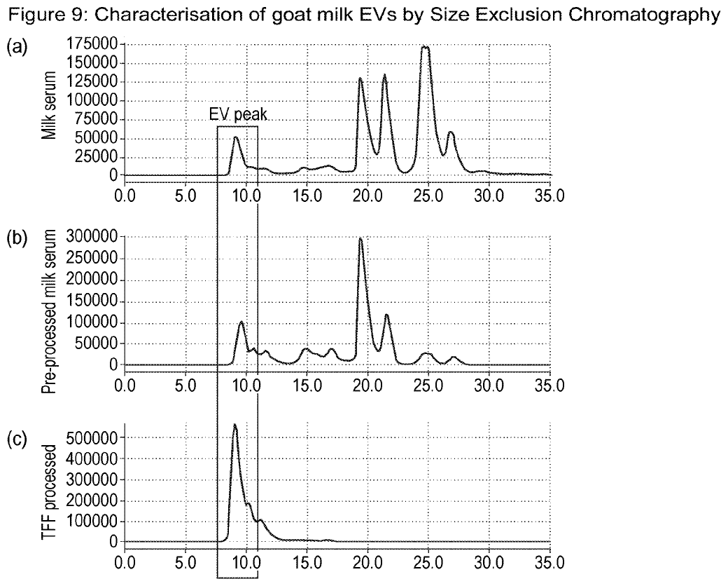

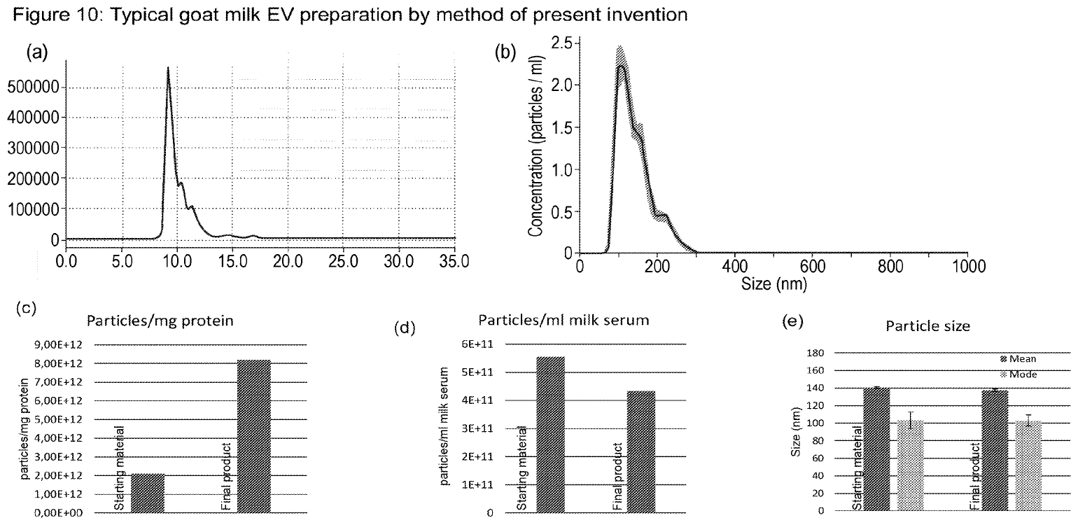

50 μL of the samples were subjected to size exclusion chromatography using a Superdex 200 10/300 (Cytiva) column on a Shimadzu LC-20Ai system equipped with a SPD-M20A PDA Diode Array Detector and a RF-20A Fluorescence Detector. Size exclusion was performed in PBS pH 7.4 at a flow rate of 0.8 mL/min. Size of eluted peaks was determined using a gel filtration standard (BioRad #5119011, FIG. 4). Characterisation of Milk EV preparations by methods of the present invention using SEC is illustrated in FIGS. 3, 4, 6 (a), 9, 10 (a), and 12(a).

### Example 15: Protein Analysis of EV Preparation

Protein concentrations of EV samples were either determined by Bradford (Pierce Detergent Compatible Bradford Assay Kit, Thermo Fisher Scientific 23246) or BCA (Pierce BCA Protein Assay Kit, Thermo Fisher Scientific 23225) reagents according to the manufacturer's instructions, using serial dilutions of BSA as a standard. Protein concentrations were determined in triplicates.

### Example 16: Western Blot Analysis of EV Preparations

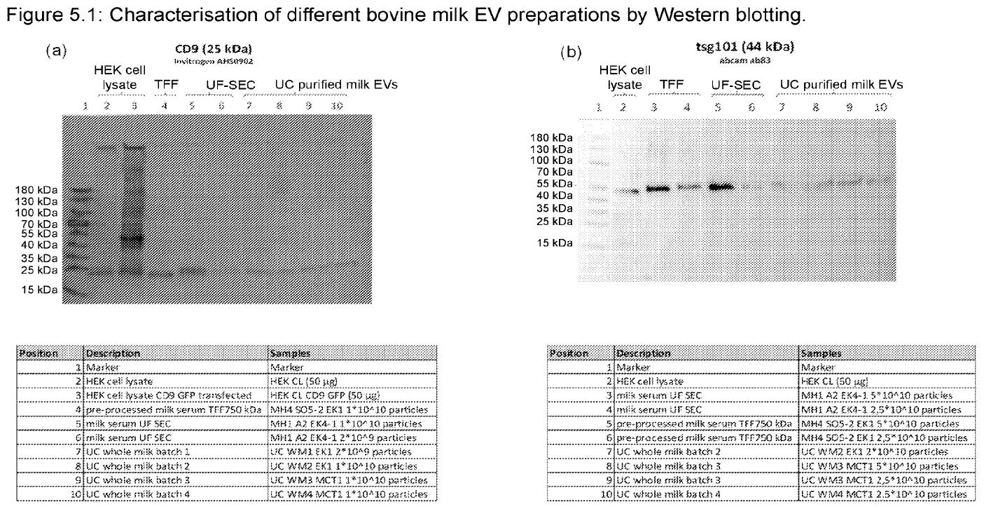

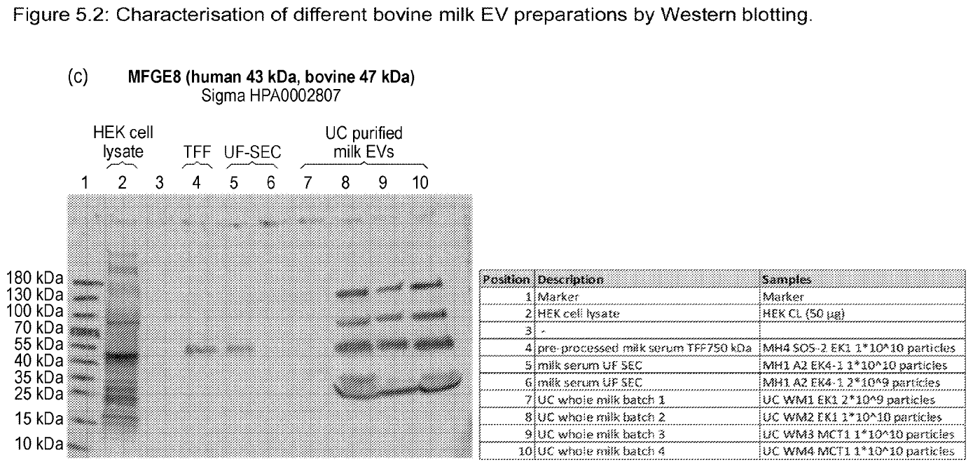

EV samples were analysed by SDS-PAGE using 4-20% TGX Gels (BioRad) under reducing (for markers MFGE8, tsg101) or non-reducing (CD9) conditions at 150 Volt for approximately 50 min. Separated proteins were transferred to a 0.45 μM nitrocellulose membrane by semi-dry blotting at 20 Volt for 45 min. Blocking was either performed using TBS+0.2% Tween-20+5% BSA (for markers MFGE8, CD9) or TBS+0.2% Tween-20+2% non-fat dry milk (tsg101) for 1 hour at RT while shaking. After blocking, membranes were incubated at 4° C. o/n with the following primary antibodies: MFGE8 (HPA002807, Sigma, 1:1000 dilution), tsg101 (ab83, Abcam 1:1000 dilution) or CD9 (AHS0902, Invitrogen, 1:2000 dilution) diluted either in TBS+0.2% Tween-20+1% BSA (MFGE8, CD9) or TBS+0.2% Tween-20+2% non-fat dry milk (tsg101) while shaking. After incubation, the membrane was washed 5 times with TBS+0.2% Tween-20 for 5 min each and subsequently incubated with the corresponding secondary antibody (LI-COR) for 1 hour at RT while shaking (1:15000 anti-mouse IRDye 680RD to detect tsg101 and CD9 or 1:15000 anti-rabbit IRDye 800 CW to detect MFGE8). Membranes were washed 5 times for 5 minutes with TBS+0.2% Tween-20 before analysis on a LI-COR imaging instrument. Characterisation of Milk EV preparations by methods of the present invention using Western Blots is illustrated in FIG. 5.

### Example 17: Cell Uptake after EV Labelling

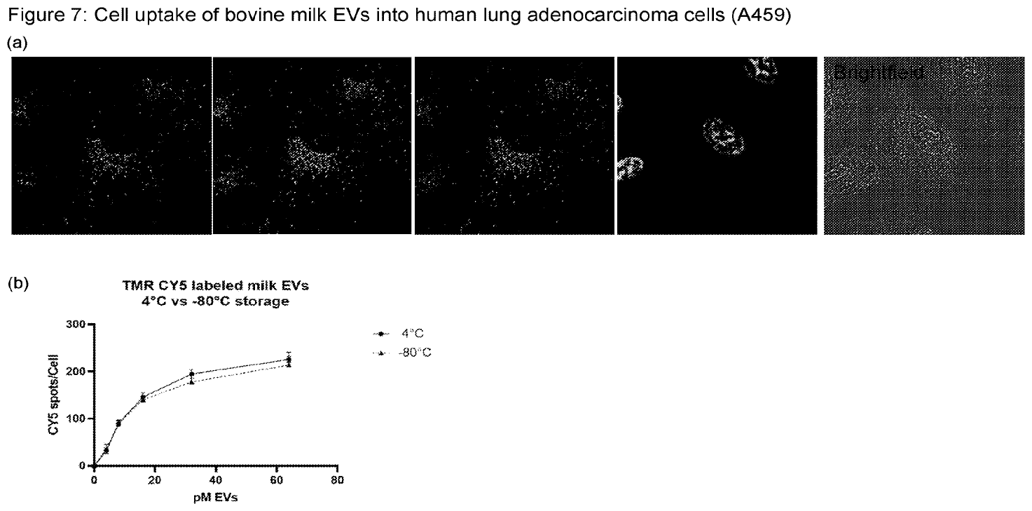

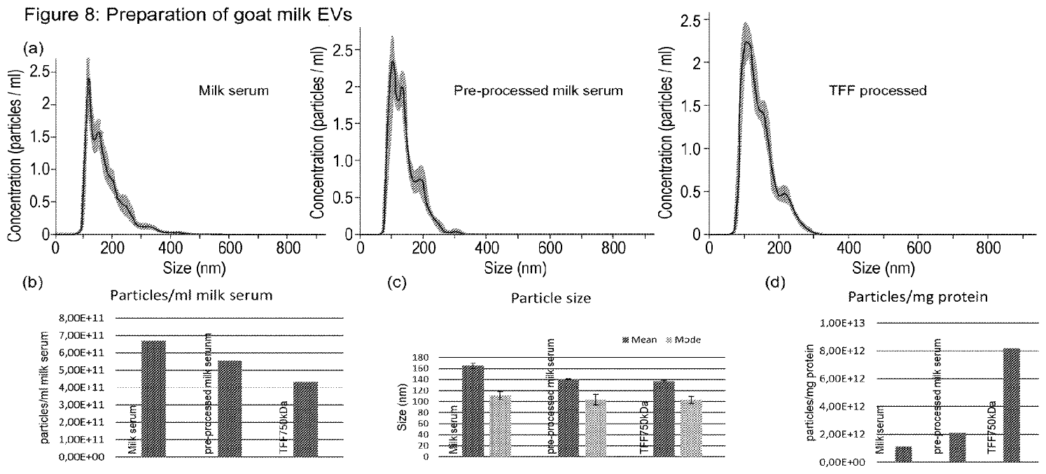

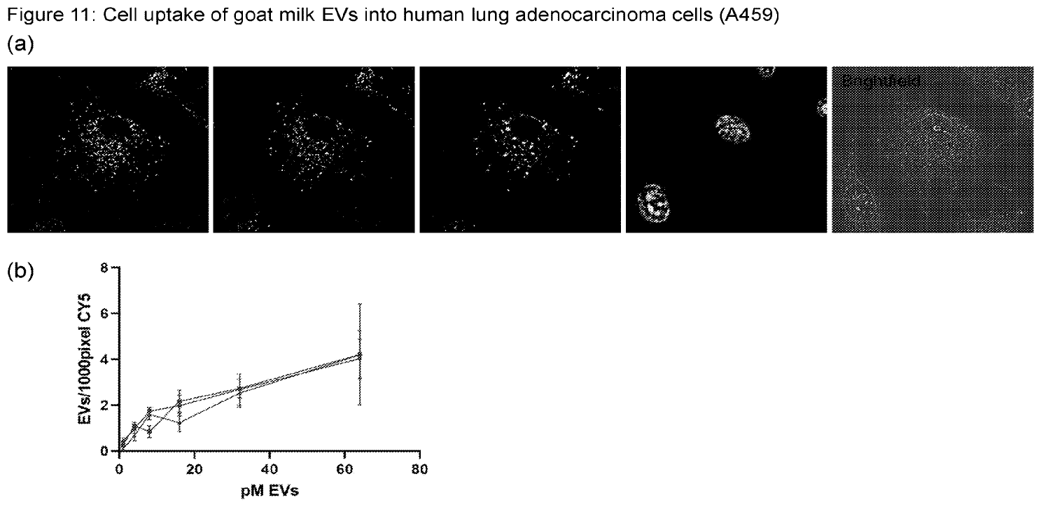

Milk EVs obtained in Example 8 as well as Example 11 were each diluted to 10 UM in PBS pH 7.4 and reacted with 25 μM (5′,6′-)TMR-NHS and 50 μM Cy5-NHS for 1 h at 37° C. Nonreacted dye was removed by six concentration/dialysis cycles with each time six volumes of PBS pH 7.4 over a hollow-fibre 750 kDa mPES membrane as described in Example 5. EV concentrations were determined by NTA as described in Example 13. A549 (human lung adenocarcinoma) cells were plated in collagen precoated 96 well plates and grown under standard cell culture conditions to a cell confluency of 50-60% (5% CO₂, 95% humidity, 37° C.). Cy5/TMR double labelled extracellular vesicles (bovine milk serum derived EVs stored at 4° C. or −80° C. (FIG. 7) or goat milk serum derived EVs stored at 4° C. (FIG. 11) were added at increasing concentrations, with three independent biological replicates per condition. After 6 hours incubation, the cells were fixed with PenFix (Richard Allen Scientific) supplemented with Hoechst 33258 at 0.5 μM. Cells were imaged by fluorescence widefield microscopy (Olympus IX83) using a 40× ApoPlan objective with a numerical aperture of 0.95. Per well over 10 images were taken, whereas every image represented a maximum intensity projection of 7 individual Z-images and 5 different channels were imaged: Brightfield, Hoechst (ex385 nm, em417-477 nm), TMR (ex545 nm, em605/70 nm), CY5 (ex640 nm, em700/75 nm) and TMR-CY5 FRET (ex545, em700/75). Cell and vesicle detection was performed with a self-programmed ImageJ Fiji plugin. Briefly, cells were detected by edge detection of the brightfield channel followed by smoothening and applying a threshold on the image. EVs were detected by usage of the Li auto threshold (similar thresholds are used in all concentrations) on the CY5 channel. EVs in the cell area were counted and are shown as “EVs/1,000 pixel” cell area.

### Example 18: Transmission Electron Microscopy of EVs with Negative Stain

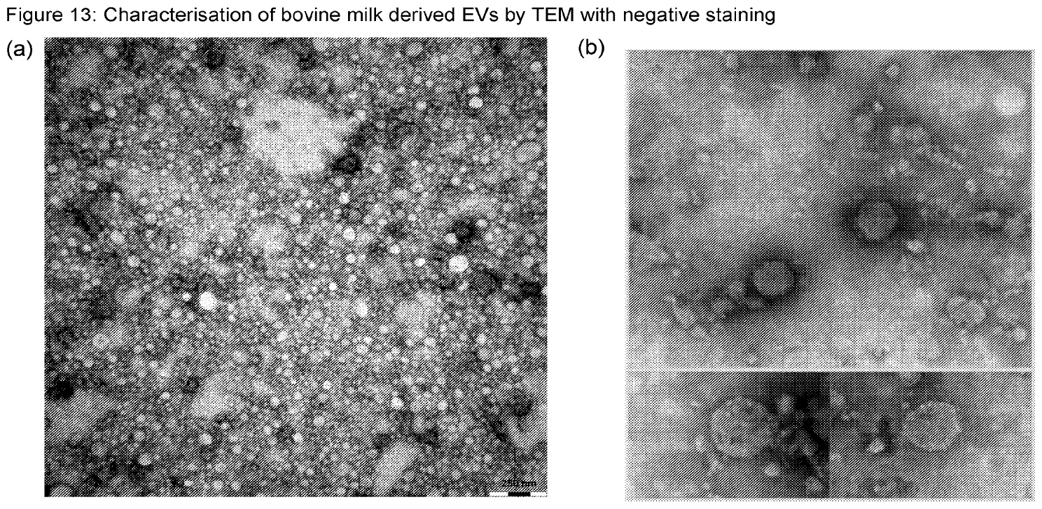

Milk EVs obtained in Example 8 were diluted to a concentration of 1 to 5×10\hat{ }10 particles per mL in PBS and pipetted onto Formvar and carbon coated grids. After air drying for 30-45 min at room temperature, excess of the EV solution was removed by pulling the grids over filter paper at an angle of ca. 45°. The grids were then washed 3× with distilled water by placing them on 100 μL drops on parafilm, followed by fixation with 1% glutaraldehyde in PBS for 5 minutes on 40 μL droplets, 8× washing with water in 40 μL droplets and negative staining with aqueous 2% uranyl acetate for 5 minutes. The samples were then air dried for 1 hour and imaged on a Zeiss, EM 910 Transmission Electron Microscope at 80,000 kV and 40,000× magnification (Tröndle camera). Typical images of milk EV preparations from Example 8 are shown in FIG. 13.

### Example 19: Quantification of (Milk EV Loaded) Amphotericin B Using High-Performance Liquid Chromatography

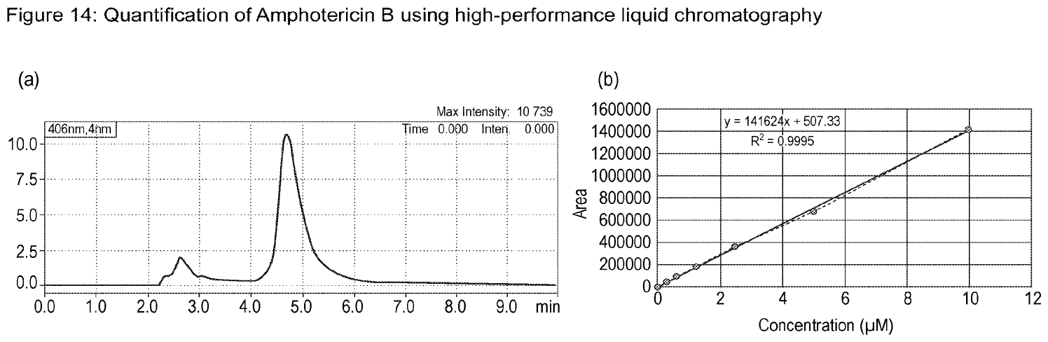

The quantification of Amphotericin B (AmB) was carried out using reversed phase high-performance liquid chromatography (RP-HPLC) on a Shimadzu Prominence instrument. Loaded MEV samples were 50 μL of samples were injected and separated on a C18 column (3.0×150 mm with particle sizes of 5 μm) by isocratic elution with a mobile phase of Acetonitrile/20 mM EDTA (55%/45% vol/vol) with a flow rate of 1 mL/min. EDTA was used in the mobile phase as it improves the chromatographic behaviour of AmB by direct competition against amphotericin B for chelation with metal ions. AmB was detected by measuring UV absorption at 406 nm Calibration standard solutions were prepared at concentrations ranging from 0.0125-10 μg/mL. Representative chromatograms and the AmB calibration curves are shown in FIG. 14 a and b, respectively.

### Example 20: Loading of Amphotericin B into Bovine Milk Exosomes

For loading of AmB into bovine milk exosomes, several methods were tested, including incubation at room temperature, incubation at 37° C., hypotonic condition, sonication, extrusion, incubation with saponin, and freeze thaw. AmB was added from a stock solution of 1 mM in DMSO to milk EV samples in PBS to final concentrations of MEVs at 1 nM, AmB at 2 μM and DMSO at 0.5% and treated under different conditions as follows:
Incubation: the formulations were incubated at room temperature (21° C.), 37° C. or 50° C. for different times (as specified in Table 1) in PBS.
Saponin: saponin was added at a final concentration of 2% w/v and the formulations were incubated at different conditions (as specified in Table 1).
Sonication: the formulations were sonicated using a probe sonicator (80% power with 30 seconds pulse/30 seconds pause).
Hypotonic condition: the formulations were diluted with H₂O to a final concentration of 0.5×PBS.
Freeze thaw: the formulations were shock frozen, kept at at −80° C. for 0.5 h and thawed at room temperature for 3 cycles.
Extrusion: the formulations were extruded (×10 times and ×20 times) using an Avanti Lipids extruder with a pore diameter of 200 nm.

To remove free AmB from the formulation, the mixture ultrafiltrated six times with PBS by centrifugation in Vivaspin ultrafiltration units (MWCO 10 kDa) at 10,000×g for 15 minutes. The final washing step resulted in 10-fold concentrated samples. AmB was then extracted by adding acetonitrile to the original volume, resulting in a final concentration of ACN H₂O of 90:10. The mixture was vortexed at room temperature at 1,000 rpm for 30 minutes and centrifuged at 1,000×g for 15 minutes prior to injection for determination of the AmB concentration using RP-HPLC as described in Example 19. The concentrations of AmB retained for the different conditions is listed in Table 1, revealing that loading by incubation at room temperature for >6 h resulted in the highest loading densities.

TABLE 1
Concentration of AmB retained in bovine milk
exosomes under different loading conditions.

| Condition | AmpB Retained | |
|---|---|---|
| Room Temperature 1 h | 0 | nM |
| Room Temperature 2 h | 21 | nM |
| Room Temperature 4 h | 179 | nM |
| Room Temperature 6 h | 227 | nM |
| Room Temperature 24 h | 252 | nM |
| 37° C. 6 h | 171 | nM |
| 37° C. 24 h | 128 | nM |
| 50° C. 6 h | 87 | nM |
| 50° C. 24 h | 52 | nM |
| Hypotonic condition | 189 | nM |
| Sonication 2.5 min (ice bath) | 294 | nM |
| Sonication 5 min (ice bath) | 162 | nM |
| Sonication 10 min (ice bath) | 81 | nM |
| Sonication 2.5 min (without ice bath) | 44 | nM |
| Sonication 5 min (without ice bath) | 24 | nM |
| Extrusion 10 times | 184 | nM |
| Extrusion 20 times | 172 | nM |
| Room Temperature 24 h + Saponin 0.2% | 254 | nM |
| 37° C. 24 h + Saponin 0.2% | 132 | nM |
| 50° C. 24 h + Saponin 0.2% | 54 | nM |
| Freeze Thaw 3 cycles | 108 | nM |

### Example 21: Loading of Amphotericin B into Goat Milk Exosomes

AmB loading into goat milk exosomes was tested using procedures as described in Example 20. The concentration of AmB retained was carried out using RP-HPLC as described in Example 19.

TABLE 2
Concentration of AmB retained in goat milk
EVs (1 nM) at different conditions.

| Condition | AmB Retained | |
|---|---|---|
| Room Temperature 2 h | 82 | nM |
| Room Temperature 4 h | 271 | nM |
| Room Temperature 6 h | 392 | nM |
| Room Temperature 24 h | 388 | nM |
| 37° C. 6 h | 199 | nM |
| 37° C. 24 h | 112 | nM |
| 50° C. 6 h | 91 | nM |
| 50° C. 24 h | 48 | nM |
| Sonication 2.5 min (ice bath) | 209 | nM |
| Sonication 5 min (ice bath) | 103 | nM |
| Sonication 10 min (ice bath) | 73 | nM |

### Example 22: Saturation of AmB Loading into Goat Milk EVs

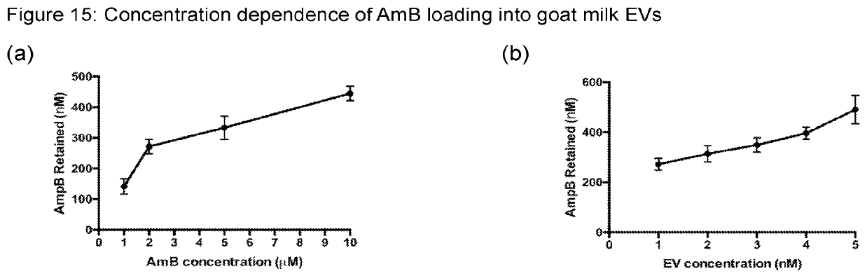

5 To additionally test the saturation of EV loading as a function of AmB concentration, goat milk EVs at 1 nM were incubated for 6 h at room temperature with increasing concentrations of AmB ranging from 1 μM to 10 μM (DMSO at 1% v/v in all samples). Furthermore, we tested the formulation of AmB-loaded exosomes at increasing EV concentration while keeping the EV:AmB ratio constant. The MEV:AmB concentrations were at 1 nM:2 μM; 2 nM:4 μM; 3 nM:6 μM; 4 nM:8 UM and 5 nM:10 μM. All formulations contained 1% DMSO. The concentration of AmB retained was carried out using RP-HPLC as described in Example 19. Results are shown in FIG. 15.

### Example 23. Co-Fractionation of AmB with Milk EVs Using Size Exclusion Chromatography

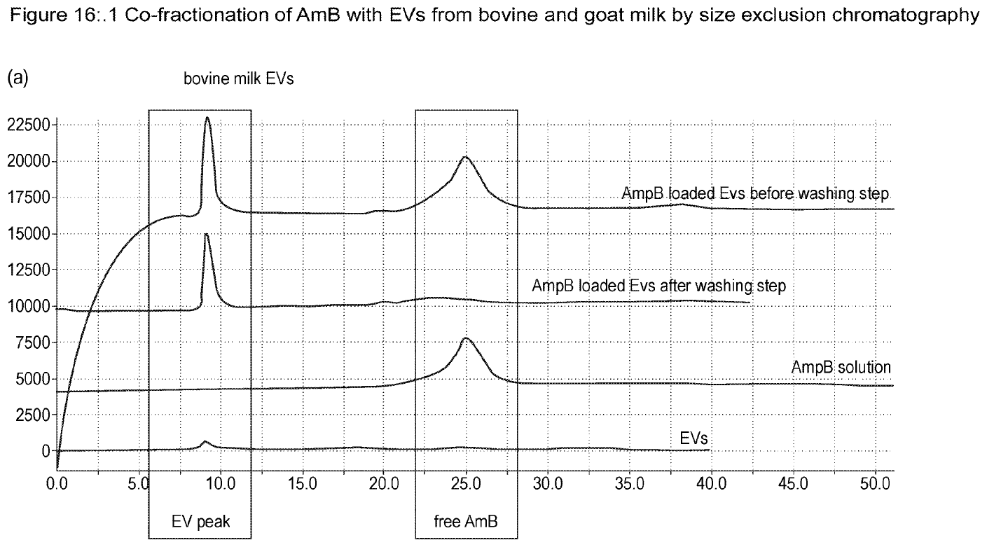

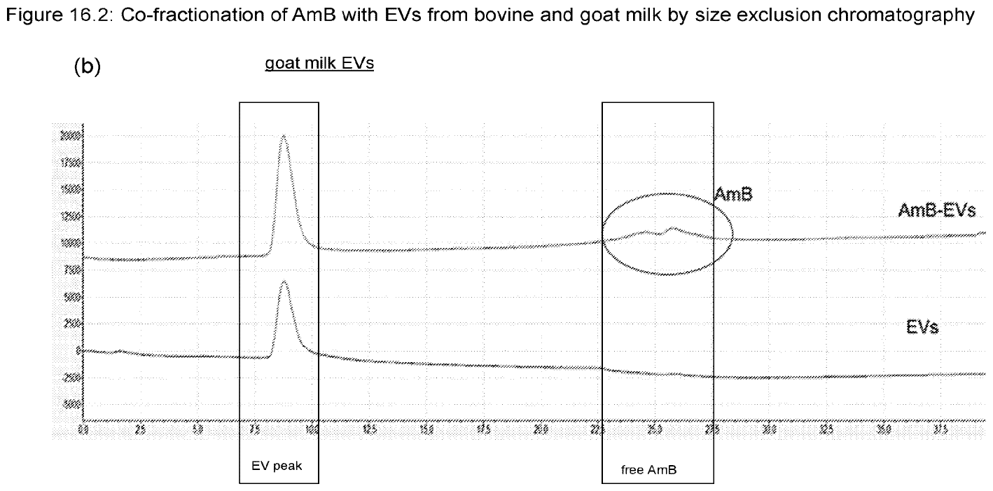

To test whether AmB retained with EVs after loading is indeed physically associated with the vesicles, we used analytical size exclusion chromatography under native conditions to test for co-fractionation. 10 μL of the samples as prepared in Example 20 (1 nM bovine or goat milk EVs loaded with 2 M AmB by incubation for 6 h at room temperature) before and after elimination of free AmB by ultrafiltration were injected onto a Sephadex 200 30/10 column and separated by isocratic elution with PBS pH 7.4 at a flow rate of 0.8 mL/min on a Shimadzu LC-20Ai instrument equipped with a diode array UV/Vis and a fluorescence detector. AmB was detected at 406 nm absorption, milk EVs were detected by autofluorescence at 488 nm/510 nm (ex/em). In addition to the formulations, free AmB and MEVs were also analysed. As shown in FIG. 16, AmB loaded in both, bovine (FIG. 16a) and goat milk EVs (FIG. 16b) also cofractionated with the MEV peak by native size exclusion chromatography, demonstrating physical association of AmB with the vesicles.

### Example 24: Cell Uptake of AmB Loaded Goat Milk EVs

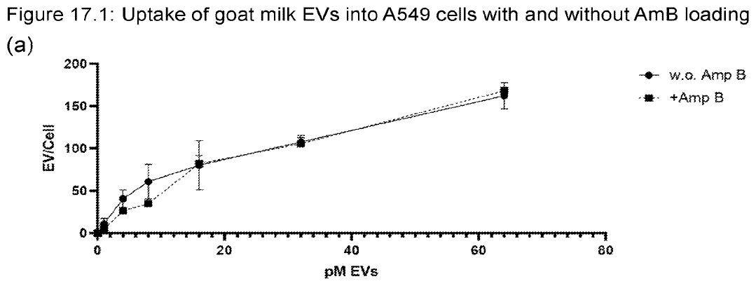

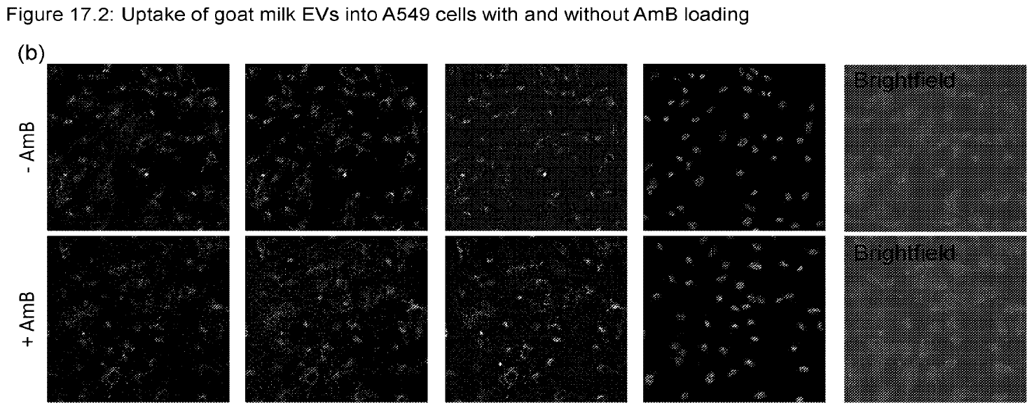

Goat milk EVs were labelled with Cy5 NHS and Tamra NHS as described in Example 17 and loaded with AmB as described in Example 21 (1 nM goat milk EVs loaded with 2 μM AmB by incubation for 6 h at room temperature). Cell uptake in A549 cells was then quantitatively by fluorescence microscopy as described in Example 17. As shown in FIG. 17, goat milk exosomes loaded with AmB were taken up by the cells identically to the non-loaded goat milk EVs, confirming that AmB loading does not affect the functionality of milk EVs in cell uptake.

### Example 25: Systemic Exposure of Milk EVs of Present Invention Upon Nasal Delivery Via the Respiratory Tract

Bovine milk EVs labelled with TMR and Cy5 obtained in Example 17 were used for intranasal administration in C57/B16 mice (female, 8 weeks). After aspiration of 5 μL of labelled EVs at 3.4×10\hat{ }13 particles/mL in PBS via the nose, mice were sacrificed after 6 h, organs were fixed with 4% PFA, transferred into a Cryobuffer (30% w/v Sucrose, 1% w/v Polyvinyl-pyrrolidone-40, 30% v/v Ethylene glycol in 100 mM PBS pH 7.4) and analysed by epifluorescence imaging on an IVIS Spectrum imaging instrument. Fluorescence signal intensities relative to organs 5 from PBS treated animals are shown in Table 3.

TABLE 3
Fluorescence signal intensities of different
organs from EV dosed mice compared to matched
organs of PBS treated control animals:
Relative signal intensity vs. control

| Tissue | Cy5 Signal Intensity |
|---|---|
| Brain | 1.2 |
| GI tract | 44.2 |
| Heart | 7.7 |
| Kidney left | 32.4 |
| Kidney right | 29.5 |
| Liver | 18.5 |
| Lymph node | 5.7 |
| Lung left | 386.0 |
| Lung right | 412.7 |
| Spleen | 7.0 |

### Example 26: Susceptibility of Candida albicans to Milk EVs Loaded with AmpB

Susceptibility of Candida albicans SC5314 to milk EVs and milk EVs loaded with Amphotericin B (AmpB) was performed by the broth microdilution method, using RPMI-1640 medium and MOPS buffer in a concentration range from 200 nM and 0.39 nM for AmpB loaded EVs and equal particles/mL of unloaded EVs. AmpB alone was also tested at concentrations from 8 UM to 15.12 nM. The MIC (Minimum Inhibitory Concentration) was recorded as the lowest concentration of the drug and EVs that produced a visible decrease in turbidity after 24 hours compared to drug-free growth control.

The broth micro dilution test is performed using sterile round bottomed 96 well microtiter plates. 50 μL of 1×PBS at pH 7.4 is added to Rows 1 to Row 7, except for Column 1 of Row 3 to Row 7. 100 μL of EVs (BMEV, BMEV-AmpB, GMEV and GMEV-AmpB) at 2× desired concentrations is added to Rows 4 to Row 7 of Column 1. 100 μL of AmpB at 2× desired concentration (i.e. 16 μM) is added to Row 3 of Column 1. 2-fold dilutions were performed by transferring 50 μL from Column 1 to Column 10 of Rows 3 to 7, and homogenised to ensure proper dilution of the compound by half. Row 1 was used as a blank for spectrophotometric readings and Row 2 is used as a growth control. After the plates are prepared, 2× inoculum concentration are prepared with a total cell concentration of 1e+3 to 5e+3 CFU/mL prepared in in RPMI 1640 media (with glutamine, without bicarbonate, and with phenol red as a pH indicator) in MOPS according to CLSI media preparation guidelines. Each well of the microtiter plate was inoculated with 50 μL of the 2× inoculum to yield a final cell concentration of 0.5e+3 to 2.5e+3 CFU/mL in the wells. After inoculation, the plates are incubated at 35° C. for 24 hours. Post-incubation, the plates are read at 600 nm using the Thermo Scientific Multiskan Go, and data was recorded. Agitation prior to reading the plate will help with accurate readings.

### Media Preparation

10.4 g powdered RPMI 1640 medium (with glutamine and phenol red, without bicarbonate); 34.53 g MOPS (3-[N-morpholino] propanesulfonic acid) buffer.

Dissolve powdered medium in 900 mL distilled H₂O. Add MOPS (final concentration of 0.165 mol/L) and stir until dissolved. While stirring, adjust the pH to 7.0 at 25° C. using 1 mol/L sodium hydroxide.

Add additional water to bring medium to a final volume of 1 L. Filter sterilize and store at 4° C. until use.

### Inoculum Preparation

The steps for preparation of inoculum are as follows:
I. Candida albicans SC5314 was subcultured from sterile vials onto Sabouraud dextrose agar or Potato Dextrose agar and passaged to ensure purity and viability. The incubation temperature throughout was 35° C.
II. The inoculum was prepared by picking five colonies of ca. 1 mm in diameter from 24-hour old culture. The colonies were suspended in 1 mL of sterile 1×PBS at pH 7.4. A 10-fold dilution was prepared in PBS. 10 μL of the diluted inoculum with equal volume of Trypan Blue was mixed and counted using a haemocytometer.
III. The suspension should be vortexed for 15 seconds and the cell density adjusted by adding sufficient sterile 1×RPMI-1640 to yield a cell concentration of 1e+3 to 5e+3 CFU/mL.

### Results

TABLE 4
Replicate 1 numerical values (test plate FIG. 18)

| | 200 nM | 100 nM | 50 nM | 25 nM | 12.5 nM | 6.25 nM | 3.125 nM | 1.56 nM | 0.78 nM | 0.39 nM | |
|---|---|---|---|---|---|---|---|---|---|---|---|
| Abs | 1 | 2 | 3 | 4 | 5 | 6 | 7 | 8 | 9 | 10 | |
| A | 0.00 | 0.00 | 0.00 | 0.00 | 0.00 | 0.00 | 0.00 | 0.00 | 0.00 | 0.00 | Blank |
| B | 0.14 | 0.13 | 0.15 | 0.15 | 0.13 | 0.16 | 0.16 | 0.19 | 0.16 | 0.16 | Growth control |
| C | 0.00 | 0.00 | 0.00 | 0.08 | 0.08 | 0.13 | 0.15 | 0.13 | 0.15 | 0.13 | AmpB |
| D | 0.47 | 0.43 | 0.33 | 0.30 | 0.24 | 0.22 | 0.20 | 0.18 | 0.17 | 0.18 | BMEV |
| E | 0.00 | 0.00 | 0.00 | 0.05 | 0.15 | 0.19 | 0.19 | 0.18 | 0.15 | 0.16 | BMEV-AmpB |
| F | 0.44 | 0.40 | 0.33 | 0.25 | 0.21 | 0.20 | 0.18 | 0.17 | 0.16 | 0.17 | GMEV |
| G | 0.00 | 0.00 | 0.00 | 0.00 | 0.10 | 0.17 | 0.19 | 0.17 | 0.17 | 0.17 | GMEV-AmpB |
| | 8 μM | 4 μM | 2 μM | 1 μM | 500 nM | 250 nM | 125 nM | 62.5 nM | 31.25 nM | 15.12 nM | AmpB concentration |

The above table 4 shows the numerical values for the MICs of a typical test plate obtained for one exemplary investigated strains of Candida albicans (Candida albicans SC5314). The corresponding visual image is reproduced in FIG. 18a, while FIG. 18b shows the statistical evaluation. From these results it can be seen that, surprisingly, the MIC values obtained for the Amphotericin loaded milk EVs are about 40-80× lower as compared to unformulated Amphotericin B, demonstrating the surprisingly high efficiency of this formulation.

### Example 27: Preparation of Milk EVs from Frozen Bovine Colostrum

This example proceeds like Examples 1 to 5, starting from frozen bovine colostrum (820 mL) to obtain 50 mL of purified colostrum EVs.

The colostrum was thawed slowly overnight at 4° C. Then the colostrum was subjected to a defatting step by centrifugation at 13.000×g for 30 minutes and the top layer was discarded. The defatted colostrum was then pre-processed following the steps described in Examples 1-4. The material was then subjected to a second concentration/dialysis cycle using a hollow-fibre membrane with a 750 kDa MWCO. The material was first concentrated four times to a volume of 50 mL. Then a total of 20 volumes were exchanged by dialysis by repeatedly adding 112 mL of PBS pH 7.4. Finally, the sample was concentrated to obtain ca. 50 mL of purified and concentrated bovine colostrum derived EVs. The material was sterile filtered using a 0.2 μM PES syringe filter. The resulting material was characterized with respect to particle numbers and size distribution as well as total protein content as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 9.29+11 | 5.68E+9 | 164 | 1.5 | 133 | 8.9 | 7.29 | — | 2.1E+11 | 9.3E+11 |

### Example 28: Preparation of Milk EVs from Human Breast Milk

This example proceeds like Examples 1 to 5, starting from human breast milk (70 ml) to obtain 10 mL of purified human milk EVs.

The human breast milk was thawed slowly overnight at 4° C. Then the milk was subjected to a defatting step by centrifugation at 13000×g for 30 min and the top layer was discarded. Then the defatted human milk (60 ml) was heated to 37° C. and CaCl₂) (100 mg/100 mL milk) and rennet (8 mg/100 mL milk) were added. The human breast milk was then pre-processed following the steps described in Examples 1-4. The pre-processed material (20 mL) was then subjected to a second concentration/dialysis cycle using a hollow-fibre membrane with a 750 kDa MWCO, exchanging 6×40 mL PBS and finally concentrating the material to 10 mL. The material was sterile filtered using a 0.2 μM PES syringe filter. The resulting material was characterized with respect to particle numbers and size distribution as well as total protein content as described in Examples 13 and 15.

| | | Size | | Size | | Protein | | | Yield |
|---|---|---|---|---|---|---|---|---|---|
| Concentration | | (nm) | | (nm) | | concentration | | Particles/mg | (particles/ml |
| Particles/ml | SD | mean | SD | mode | SD | mg/ml | SD | protein | milk serum) |
| 9.29+11 | 5.68E+9 | 164 | 1.5 | 133 | 8.9 | 7.29 | — | 2.1E+11 | 9.3E+11 |

### Example 29: Isolation of Milk EVs with Different Pore Size Membranes

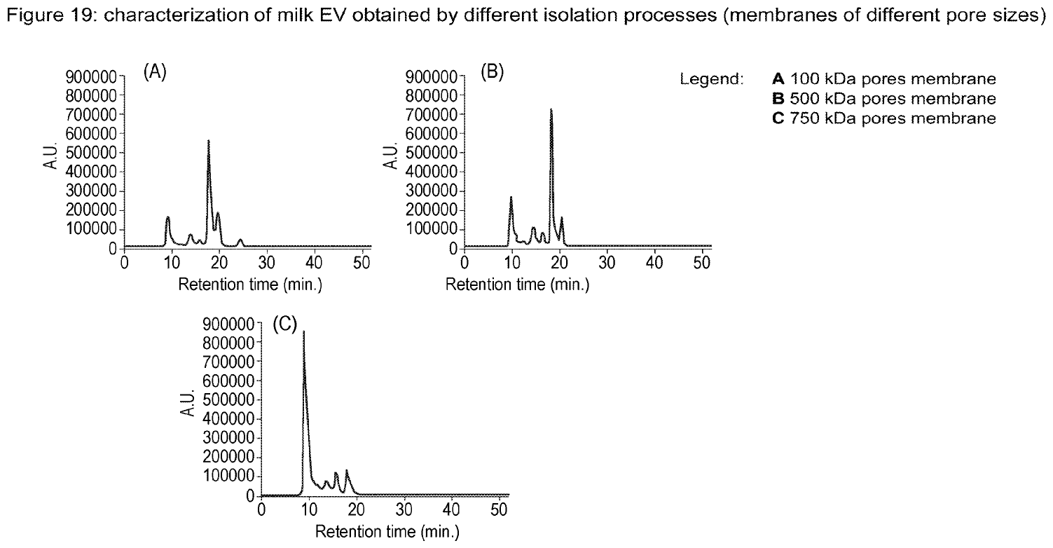

Milk EVs isolation was carried out, according to the invention as described in example 3, and then further purified in three different portions by dialysis, exchanging other 10 volumes against phosphate buffer (10 mM) using 3 different membranes, characterized by different exclusion cut-off limits. In particular, 750 kDa, 500 kDa and 100 kDa membranes were employed and the purities of the obtained milk EVs were measured by size exclusion chromatography (SEC) as described in Example 14. In FIG. 19 are reported the chromatographic graph of the three different samples, obtained always dialyzing the same volume of solutions. The results shows that the purity grade of the samples is 18% using the 100 kDa membrane (A), 27% with the 500 kDa membrane (B) and 91% using the 750 kDa membrane (C).

### Example 30: EVs Lyophilization Process

Milk EVs were isolated as described in example 9 with the modification that instead of PBS only phosphate buffer (10 mM without salt) was used for the final dialysis, which was carried out using a 750 kDA membrane. The obtained final liquid is then subjected to a lyophilization process using a standard industrial lyophilizer (Zirbus, 150 L capacity) with the following process conditions: the liquid material (about 40 L) was pre-cooled to 4° C. and loaded into the shelfs via a pump. The temperature was then lowered to −20° C. at a range of about 20° C./h. Once the product temperature reached below −20° C., a three-step drying program was executed with a first drying phase at 0.8 mbar for 24 h, then 24 h at 0.5 mbar and 24 h final drying at maximum vacuum and temperatures below 25° C. Finally, the vacuum was broken, the product harvested and immediately transferred into sealed aluminium bags. For further analysis, samples from the dried lyophilizate were reconstituted to their original concentration in pure water by using a magnetic stirrer. The reconstituted samples were then characterized measuring particle sizes and concentrations (NTA, as described in example 13, and total protein measurements as described in example 15). IR-spectra were recorded on a Bruker Alpha II instrument by taking ca 3 μL of liquid sample and placing it on the ATR crystal, letting it dry before measurement.

The stability of the milk EV preparations toward a freezing process is demonstrated in FIG. 20, where the particle concentration and the particle size measurement of a sample of milk EV upon storage at 4° C. and after freezing and thawing is reported.

The stability of the milk EV preparations toward a lyophilization process is demonstrated in FIGS. 21 and 22, where particle concentration (FIG. 21 (a—Lyo)) and particle size (FIG. 21 (b—Lyo)) as well as the IR spectrum of milk EV after reconstitution of lyophilized milk EV preparations (FIG. 22-B) are reported, in comparison with the reference samples.

### Example 31: EVs Spray-Drying Process

Milk EVs were isolated as described in example 9, with the modification that, instead of PBS, only phosphate buffer (10 mM without salts) was used for the final dialysis, which was carried out using a 750 kDA membrane. The obtained final liquid is then subjected to a spray-drying process using a standard pilot spray-dryer with a drying capacity of 2 L per hour, and using the following process parameters and conditions: the feed solution was pre-cooled at 4° C.; the inlet temperature was set to ca. 165° C., the outlet temperature to ca. 80° C. For further analysis, samples of the obtained powder were reconstituted to their original concentration in pure water by using a magnetic stirrer. The reconstituted samples were then characterized measuring particle sizes and concentrations (NTA, as described in example 13, and total protein measurements as described in example 15). IR-spectra were recorded on a Bruker Alpha II instrument by taking ca. 3 μL of liquid sample and placing it on the ATR crystal, letting it dry before measurement.

The stability of the milk EV preparations toward a heating process is demonstrated in FIG. 23, where the IR spectrum of milk EV preparations before and after heating at 95° C. is reported. The stability of the milk EV preparations toward a spray-drying process is demonstrated in FIGS. 21 and 22, where particle concentration (FIG. 21 (a—Spray)) and particle size (FIG. 21 (b—Spary)) as well as the IR spectrum of milk EV after reconstitution from a spry-dried milk EV preparation (FIG. 22-C) are reported, in comparison with the reference samples.

1. A process for isolation of purified milk EVs, said process comprising the following steps:
a) providing a sample of milk;
b) optionally clarifying said sample by means of traditionally known filtration or decantation processes, to obtain cleared milk;
c) subjecting said milk or said cleared milk to a treatment with an enzyme mix, suitable for coagulating milk proteins at a pre-defined temperature and time, to obtain a liquid, containing coagulated proteins;
c′) performing a thermal treatment on said liquid containing coagulated proteins;
d) separating said coagulated proteins from the liquid by performing at least one filtration of said liquid, with an inert layer of a filtration aid, to obtain a clear solution;
e) subjecting said clear solution to at least one ultrafiltration and concentration steps, using membranes of defined pore-sizes and ionic strength and adjusted water or buffer as dialysis medium, in order to obtain an EVs concentrated preparation;
f) optionally treating said EVs concentrated preparation with a stabilizing agent, and, concomitantly, adjusting the pH obtaining a stabilized EVs concentrated preparation;
g) subjecting said EVs concentrated preparation or said stabilized EVs concentrated preparation, to a second filtration/clarification step in order to obtain a clarified essentially pure milk EVs isolate;
h) optionally subjecting said clarified essentially pure milk EVs isolate to a sterile filtration, using standard sterile filters of pore sizes below 1 μm to obtain a clarified essentially pure milk EVs sterile isolate.

2. The process according to claim 1, wherein said milk protein coagulation step or enzyme mix of step (c) contains pepsin and chymosin.

3. The process according to claim 1, wherein said thermal treatment of step (c′) is carried out by heating said liquid containing coagulated proteins obtained in step (c) to 45-65° C.

4. The process according to claim 1, wherein said at least one ultrafiltration and concentration of step (e) is performed through a membrane having a molecular weight cut-off of 750 kDa and is preceded by a pre-concentration and ultrafiltration step through a membrane of 100-500 kDa molecular weight cut-off.

5. The process according to claim 1, wherein said clarified essentially pure milk EVs sterile isolate obtained from step (h) is further spray-dried and/or frozen, with or without one or more cryoprotectants and lyophilized.

6. A method of delivering active principles in the pharmaceutical, veterinary, nutraceutical and/or cosmetic fields with the clarified essentially pure milk EV isolate according to claim 1, said method comprising
loading said active principles on a carrier being said clarified essentially pure milk EV.

7. Composition comprising the clarified essentially pure milk EVs isolate according to claim 1, and pharmaceutically acceptable excipients.

8. Composition according to claim 7, further comprising amphotericin B loaded into said clarified essentially pure milk EVs isolate.

9. The method according to claim 6, wherein said carrier comprises clarified essentially pure milk EV and pharmaceutical acceptable excipients.

10. The method according to claim 9 for inhalation.

11. Composition according to claim 8 as antifungal and/or antiparasitic.

12. The process according to claim 1, wherein said stabilizing agent is potassium sorbate.

13. The process according to claim 1, wherein the standard sterile filters have pore sizes between 0.45 μm and 0.2 μm.

14. The process according to claim 2, wherein said coagulation step or enzyme mix of step (c) is obtained via fermentation or is rennet.

15. The process according to claim 2, wherein said coagulation step or enzyme mix of step (c) contains 5% of pepsin and 95% of chymosin.

16. The process according to claim 3, wherein said thermal treatment of step (c/) is carried out by heating said liquid at 53-56° C.
